SEQUENCE LISTING (15 sequences)

SEQ ID NO: 1 - protein, 314 aa, synthetic construct
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 2 - protein, 119 aa, synthetic construct
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 3 - protein, 20 aa, synthetic construct
GGGGSGGGGSGGGGSGGGGS

SEQ ID NO: 4 - protein, 453 aa, synthetic construct
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 5 - protein, 1130 aa, unidentified
MRCLAPRPAGSYLSEPQGSSQCATMELGPLEGGYLELLNSDADPLCLYHFYDQMDLAGEEEIELYSEPDTDTINCDQFSRLLCDMEGDEETREAYANIAELDQYVFQDSQLEGLSKDIFKHIGPDEVIGESMEMPAEVGQKSQKRPFPEELPADLKHWKPAEPPTVVTGSLLVRPVSDCSTLPCLPLPALFNQEPASGQMRLEKTDQIPMPFSSSSLSCLNLPEGPIQFVPTISTLPHGLWQISEAGTGVSSIFIYHGEVPQASQVPPPSGFTVHGLPTSPDRPGSTSPFAPSATDLPSMPEPALTSRANMTEHKTSPTQCPAAGEVSNKLPKWPEPVEQFYRSLQDTYGAEPAGPDGILVEVDLVQARLERSSSKSLERELATPDWAERQLAQGGLAEVLLAAKEHRRPRETRVIAVLGKAGQGKSYWAGAVSRAWACGRLPQYDFVFSVPCHCLNRPGDAYGLQDLLFSLGPQPLVAADEVFSHILKRPDRVLLILDGFEELEAQDGFLHSTCGPAPAEPCSLRGLLAGLFQKKLLRGCTLLLTARPRGRLVQSLSKADALFELSGFSMEQAQAYVMRYFESSGMTEHQDRALTLLRDRPLLLSHSHSPTLCRAVCQLSEALLELGEDAKLPSTLTGLYVGLLGRAALDSPPGALAELAKLAWELGRRHQSTLQEDQFPSADVRTWAMAKGLVQHPPRAAESELAFPSFLLQCFLGALWLALSGEIKDKELPQYLALTPRKKRPYDNWLEGVPRFLAGLIFQPPARCLGALLGPSAAASVDRKQKVLARYLKRLQPGTLRARQLLELLHCAHEAEEAGIWQHVVQELPGRLSFLGTRLTPPDAHVLGKALEAAGQDFSLDLRSTGICPSGLGSLVGLSCVTRFRAALSDTVALWESLQQHGETKLLQAAEEKFTIEPFKAKSLKDVEDLGKLVQTQRTRSSSEDTAGELPAVRDLKKLEFALGPVSGPQAFPKLVRILTAFSSLQHLDLDALSENKIGDEGVSQLSATFPQLKSLETLNLSQNNITDLGAYKLAEALPSLAASLLRLSLYNNCICDVGAESLARVLPDMVSLRVMDVQYNKFTAAGAQQLAASLRRCPHVETLAMWTPTIPFSVQEHLQQQDSRISLR

SEQ ID NO: 6 - protein, 295 aa, synthetic construct
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDL

SEQ ID NO: 7 - DNA, 19 bp, synthetic construct
  source 1..19: mol_type = other DNA
aagattcaggtttactcac

SEQ ID NO: 8 - protein, 434 aa, synthetic construct
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDL

SEQ ID NO: 9 - DNA, 5798 bp, synthetic construct
  source 1..5798: mol_type = other DNA
atgtctcgctccgtggccttagctgtgctcgcgctactctctctttctggcctggaggctatccagcgtgagtctctcctaccctcccgctctggtccttcctctcccgctctgcaccctctgtggccctcgctgtgctctctcgctccgtgacttcccttctccaagttctccttggtggcccgccgtggggctagtccagggctggatctcggggaagcggcggggtggcctgggagtggggaagggggtgcgcacccgggacgcgcgctacttgcccctttcggcggggagcaggggagacctttggcctacggcgacgggagggtcgggacaaagtttagggcgtcgataagcgtcagagcgccgaggttgggggagggtttctcttccgctctttcgcggggcctctggctcccccagcgcagctggagtgggggacgggtaggctcgtcccaaaggcgcggcgctgaggtttgtgaacgcgtggaggggcgcttggggtctgggggaggcgtcgcccgggtaagcctgtctgctgcggctctgcttcccttagactggagagctgtggacttcgtctaggcgcccgctaagttcgcatgtcctagcacctctgggtctatgtggggccacaccgtggggaggaaacagcacgcgacgtttgtagaatgcttggctgtgatacaaagcggtttcgaataattaacttatttgttcccatcacatgtcacttttaaaaaattataagaactacccgttattgacatctttctgtgtgccaaggactttatgtgctttgcgtcatttaattttgaaaacagttatcttccgccatagataactactatggttatcttctgcctctcacagatgaagaaactaaggcaccgagattttaagaaacttaattacacaggggataaatggcagcaatcgagattgaagtcaagcctaaccagggcttttgcgggagcgcatgccttttggctgtaattcgtgcatttttttttaagaaaaacgcctgccttctgcgtgagattctccagagcaaactgggcggcatgggccctgtggtcttttcgtacagagggcttcctctttggctctttgcctggttgtttccaagatgtactgtgcctcttactttcggttttgaaaacatgagggggttgggcgtggtagcttacgcctgtaatcccagcacttagggaggccgaggcgggaggatggcttgaggtccgtagttgagaccagcctggccaacatggtgaagcctggtctctacaaaaaataataacaaaaattagccgggtgtggtggctcgtgcctgtggtcccagctgctccggtggctgaggcgggaggatctcttgagcttaggcttttgagctatcatggcgccagtgcactccagcgtgggcaacagagcgagaccctgtctctcaaaaaagaaaaaaaaaaaaaaagaaagagaaaagaaaagaaagaaagaagtgaaggtttgtcagtcaggggagctgtaaaaccattaataaagataatccaagatggttaccaagactgttgaggacgccagagatcttgagcactttctaagtacctggcaatacactaagcgcgctcaccttttcctctggcaaaacatgatcgaaagcagaatgttttgatcatgagaaaattgcatttaatttgaatacaatttatttacaacataaaggataatgtatatatcaccaccattactggtatttgctggttatgttagatgtcattttaaaaaataacaatctgatatttaaaaaaaaatcttattttgaaaatttccaaagtaatacatgccatgcatagaccatttctggaagataccacaagaaacatgtaatgatgattgcctctgaaggtctattttcctcctctgacctgtgtgtgggttttgtttttgttttactgtgggcataaattaatttttcagttaagttttggaagcttaaataactctccaaaagtcataaagccagtaactggttgagcccaaattcaaacccagcctgtctgatacttgtcctcttcttagaaaagattacagtgatgctctcacaaaatcttgccgccttccctcaaacagagagttccaggcaggatgaatctgtgctctgatccctgaggcatttaatatgttcttattattagaagctcagatgcaaagagctctcttagcttttaatgttatgaaaaaaatcaggtcttcattagattccccaatccacctcttgatggggctagtagcctttccttaatgatagggtgtttctagagagatatatctggtcaaggtggcctggtactcctccttctccccacagcctcccagacaaggaggagtagctgccttttagtgatcatgtaccctgaatataagtgtatttaaaagaattttatacacatatatttagtgtcaatctgtatatttagtagcactaacacttctcttcattttcaatgaaaaatatagagtttataatattttcttcccacttccccatggatggtctagtcatgcctctcattttggaaagtactgtttctgaaacattaggcaatatattcccaacctggctagtttacagcaatcacctgtggatgctaattaaaacgcaaatcccactgtcacatgcattactccatttgatcataatggaaagtatgttctgtcccatttgccatagtcctcacctatccctgttgtattttatcgggtccaactcaaccatttaaggtatttgccagctcttgtatgcatttaggttttgtttctttgttttttagctcatgaaattaggtacaaagtcagagaggggtctggcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgggtggaggtggaagtggtggaggtggaagtggtggaggtggaagtggtggaggtggaagtggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattga

SEQ ID NO: 10 - DNA, 1305 bp, synthetic construct
  source 1..1305: mol_type = other DNA
atgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggaggtggaagtggtggaggtggaagtggtggaggtggaagtggtggaggtggaagtggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggagtaaggagggagatggaggcatcatgtctgttagggaaagcaggagcctctctgaagacctttaa

SEQ ID NO: 11 - DNA, 19 bp, synthetic construct
  source 1..19: mol_type = other DNA
gggaggcttatgccaatat

SEQ ID NO: 12 - DNA, 2730 bp, synthetic construct
  source 1..2730: mol_type = other DNA
atgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggaggtggaagtggtggaggtggaagtggtggaggtggaagtggtggaggtggaagtggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagatggaagcggagctactaacttcagcctgctgaagcaggctggagacgtggaggagaaccctggacctatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggaggtggaagtggtggaggtggaagtggtggaggtggaagtggtggaggtggaagtggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggagtaaggagggagatggaggcatcatgtctgttagggaaagcaggagcctctctgaagacctttaa

SEQ ID NO: 13 - protein, 909 aa, synthetic construct
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSDGSGATNFSLLKQAGDVEENPGPMSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDL

SEQ ID NO: 14 - DNA, 2730 bp, synthetic construct
  source 1..2730: mol_type = other DNA
atgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggaggtggaagtggtggaggtggaagtggtggaggtggaagtggtggaggtggaagtggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggagtaaggagggagatggaggcatcatgtctgttagggaaagcaggagcctctctgaagaccttggaagcggagctactaacttcagcctgctgaagcaggctggagacgtggaggagaaccctggacctatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggtggaggtggaagtggtggaggtggaagtggtggaggtggaagtggtggaggtggaagtggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattga

SEQ ID NO: 15 - protein, 909 aa, synthetic construct
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDLGSGATNFSLLKQAGDVEENPGPMSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

(19) United States
(12) Patent Application Publication
(10) Pub. No.: US 2026/0347766 A1
(43) Pub. Date: Nov. 5, 2026

(54) Immunocompatible Human Pluripotent Stem Cell, Method for Preparing Same, and Use Thereof

(71) Applicants: Shanghai Institute of Nutrition and Health, Chinese Academy of Sciences, Shanghai (CN); Shanghai East Hospital, School of Medicine, Tongji University, Shanghai (CN); Tong Ji University, Shanghai (CN)

(72) Inventors: Huangtian Yang, Shanghai (CN); Peng Zhang, Shanghai (CN); Senle Rao, Shanghai (CN); Xiaoqing Zhang, Shanghai (CN); Ling Liu, Shanghai (CN)

(21) Appl. No.: 18/879,327

(22) PCT Filed: Jun. 29, 2023
(86) PCT No.: PCT/CN2023/104109

§ 371 Date: Dec. 27, 2024

(30) Foreign Application Priority Data
Jun. 29, 2022 (CN) 202210752354.6

(51) Int. Cl. C12N 5/074; A61K 35/34; A61K 35/44; C07K 14/705; C07K 19/00; C12N 5/071; C12N 5/077; C12N 9/22; C12N 15/10; C12N 15/113; C12N 15/867

(52) U.S. Cl. CPC C12N 5/0607; A61K 35/34; A61K 35/44; C07K 14/70539; C07K 19/00; C12N 5/0657; C12N 5/069; C12N 9/226; C12N 15/102; C12N 15/1138; C12N 15/867; C07K 2319/00; C12N 2310/20; C12N 2510/00; C12N 2740/15043

(57) ABSTRACT

Provided are an immunocompatible human pluripotent stem cell, a method for preparing same, and use thereof. The modified human pluripotent stem cell can be induced and differentiated into various tissues/organ cells, has very ideal immunocompatibility, and has a wide application environment.

The disclosure claims the priority of Patent Application CN202210752354.6, named “Immunocompatible human pluripotent stem cell, method for preparing same, and use thereof” and filed Jun. 29, 2022, with its entire content incorporated herein by reference.

### FIELD OF THE INVENTION

The disclosure relates to the field of biotechnology, in particular, the disclosure relates to an immunocompatible human pluripotent stem cell that can escape transplant rejection, method for preparing same, and use thereof.

### BACKGROUND OF THE INVENTION

Cell transplantation therapy is a therapy of transplanting cells with specific and powerful functions that are obtained by bioengineering methods and/or treated with in vitro amplification, special culture or other treatments from original cells with specific functions into the patient's body to repair, replenish, replace, regulate or remove damaged or diseased cells/tissues, ultimately aiming to cure the disease. Therefore, the cell therapy can be used to treat various diseases, including many diseases refractory to traditional therapies, such are neurodegenerative diseases, bone disorders, diabetes, cardiovascular diseases, degenerative and traumatic diseases, or offer a complementary approach with traditional therapies. The cell therapy has a broad potential for application. However, transplant recipients can develop immune rejection against allogeneic cells, making it difficult for transplanted cells to survive in the recipient's body for a long time. Even when receiving donor organs or tissues that are HLA (human leukocyte antigen) matched, complete HLA matching is often not achievable. As a result, recipients still need to take immunosuppressive drugs for a long time, which increases the patient's burden and also brings about infection/cancer risk.

The essence of immune rejection is an immune response induced by allogenic antigens on cell surface, primarily mediated by HLA in humans. HLA is extremely polymorphic and HLA matching can reduce the immune rejection of transplanted cells by transplant recipients. Based on the immune rejection mechanism, the following methods are available to reduce immune rejection:
1. HLA matching. It is mainly used in organ and hematopoietic stem cell transplantation to reduce rejection through donor/recipient HLA matching. However, due to the scarcity of suitable donors and the extremely high polymorphism of HLA, it is very difficult to find suitable donors and meet the needs of organ transplant patients.
2. Autologous induced pluripotent stem cells (iPSCs). iPSCs are derived from reprogrammed autologous somatic cells and have the same HLA genes as the donor. In theory, iPSCs and their derived cells can also escape autoimmune rejection. However, the preparation of autologous iPSCs and their derived cells is time-consuming, difficult to control in quality, and costly. This makes them less applicable to diseases with narrow therapeutic windows and time-sensitive treatments and only applicable to a limited number of patients. Moreover, for patients with genetic diseases, the source cells of iPSCs may have functional abnormalities, leading to safety concerns. Additionally, many degenerative and traumatic diseases are common in older populations (e.g., myocardial infarction, Parkinson's disease), and it is more challenging to generate iPSCs from these individuals.
3. Establishing an HLA-matched iPSC library for general population. Due to the high polymorphism of HLA, establishing and maintaining an iPSC library with HLA matching is costly and time-consuming, making it difficult to apply in the short term. Furthermore, even tissue cells derived from different iPSC lines, such as cardiomyocytes, may exhibit differences, and using heterogeneous cells could negatively impact subsequent treatments.

All of the above methods have the drawbacks of difficulty in obtaining, high cost, and challenging quality control. Therefore, the development of universal, low-immunogenic donor cells is an urgent need in this field.

Through a review of currently published documents and patents, several approaches have been identified to obtain universal cells, including: (1) By knocking out a key HLA molecule on a chromosome, pseudo-HLA homozygous cells are constructed to increase the universality of the obtained cells; (2) By knocking out HLA I family HLA-A, -B, -C and HLA II family molecules, CD47 molecules, membrane-localized HLA-G molecules and T cell inhibitory factor PD-L1 are overexpressed with “don't eat me” signal to escape the recognition and killing effect of immune cells; (3) By completely knocking out HLA I family and HLA II family molecules, CD47 molecules are overexpressed with a “don't eat me” signal to escape the recognition and killing of T cells and macrophages; (4) By knocking out the classic HLA I molecules that mediate antigen presentation and overexpressing immunosuppressive HLA-G1 and secretory HLA-G5 molecules, the recognition and killing effect of cytotoxic T cells and natural killer cells (NK cells) can be escaped, as described in the patent application (CN113528448A) filed by the co-inventor of the disclosure Zhang Xiaoqing et al. Comparing the above four technical routes, the approach (1) is not only extremely challenging, requiring precise manipulation of individual alleles of multiple HLA molecules, but also increases the risk of genomic mutations; the approach (2) necessitates large-scale genomic deletions (greater than 13 kb) to achieve knockout of HLA-A, -B, and -C, leading to potential genomic instability; the approach (3) relies on a single immune-suppressive signal, mainly targeting macrophages. In contrast, HLAG has a broad-spectrum immunosuppressive effects and may be a better choice. Different immunosuppressive factors target different immune cells and exhibit varying effects. Therefore, the selection of appropriate immune-suppressive factor is critical to different technical approaches. The co-inventors of the disclosure constructed a B2Mm/sHLAG embryonic stem cell (hESC) line by simultaneously overexpressing membrane-localized HLA-G1 and soluble HLA-G5, thereby reducing the immunogenicity of human embryonic stem cells. This work focused on embryonic stem cells and did not specifically address human pluripotent stem cells (hPSCs), and it paves a way for further expanding its applications.

### SUMMARY OF THE INVENTION

The disclosure provides an immunocompatible human pluripotent stem cell, a method for preparing same, and use thereof, wherein the cell has a wider range of applications.

In the first aspect, the disclosure provides a method for preparing immunocompatible human pluripotent stem cells, comprising modifying human pluripotent stem cells to:
(a) not express free B2M protein, express (comprising overexpress) HLA-G1 and secreted HLA-G5; and (b) not express CIITA protein.

In one or more embodiments, in (a), the genome of the human pluripotent stem cells is modified by fusing a nucleotide encoding HLA-G1 with the endogenous B2M gene in human pluripotent stem cells, so as to express a B2M-HLA-G1 fusion protein and not express free B2M protein; preferably, the encoding gene for HLA-G1 is introduced into the position before the stop codon of exon 3 of the endogenous B2M gene, or the stop codon in exon 3 of the endogenous B2M gene is replaced by encoding gene for HLA-G1; more preferably, this modification is performed using gene editing methods; and

In one or more embodiments, in (a), an exogenous polynucleotide encoding B2M-HLA-G5 fusion protein is introduced into human pluripotent stem cells, wherein the B2M-HLA-G5 fusion protein comprises B2M and HLA-G5.

In one or more embodiments, a recombinant vector is used to express the B2M-HLA-G1 and B2M-HLA-G5; preferably, the recombinant vector comprises a non-viral vector or a viral vector.

In one or more embodiments, the non-viral vector comprises a site-directed knock-in system or a transposon system.

In one or more embodiments, the viral vector comprises a lentiviral vector.

In one or more embodiments, the B2M-HLA-G1 fusion protein further comprises a flexible linker peptide between B2M and HLA-G1, preferably the flexible linker peptide has a sequence as shown in SEQ ID NO: 3; and/or the B2M-HLA-G1 fusion protein sequentially comprises B2M and HLA-G1 from the N-terminus to the C-terminus; preferably, the amino acid sequence of the B2M-HLA-G1 fusion protein is as shown in SEQ ID NO: 4; more preferably, the nucleic acid sequence of the B2M-HLA-G1 fusion protein is as shown in SEQ ID NO: 9.

In one or more embodiments, the B2M-HLA-G5 fusion protein further comprises a flexible linker peptide between B2M and HLA-G5, preferably the flexible linker peptide has a sequence as shown in SEQ ID NO: 3; and/or the B2M-HLA-G5 fusion protein sequentially comprises B2M and HLA-G5 from the N-terminus to the C-terminus; preferably, the amino acid sequence of the B2M-HLA-G5 fusion protein is as shown in SEQ ID NO: 8; more preferably, the nucleic acid sequence of the B2M-HLA-G5 fusion protein is as shown in SEQ ID NO: 10.

In one or more embodiments, in (b), exon 3 of the CIITA gene in the genome of human pluripotent stem cells is knocked out; preferably, the knockout is performed by gene editing methods; more preferably, a gRNA with the nucleotide sequence shown in SEQ ID NO: 11 (GGGAGGCTTATGCCAATAT) is used for gene editing.

In one or more embodiments, the method comprises one or more of the following A-D:
A. the HLA-G1 comprises: a peptide with the amino acid sequence as shown in SEQ ID NO: 1; or a derivative peptide (comprising active fragments, active variants) having more than 90% sequence identity to SEQ ID NO: 1 and the function of the peptide with the amino acid sequence as shown in SEQ ID NO: 1;
B. the B2M comprises: a peptide with the amino acid sequence as shown in SEQ ID NO: 2; or a derivative peptide (comprising active fragments, active variants) having more than 90% sequence identity to SEQ ID NO: 2 and the function of the peptide with the amino acid sequence as shown in SEQ ID NO: 2;
C. the CIITA protein comprises: a peptide with the amino acid sequence as shown in SEQ ID NO: 5; or a derivative peptide (comprising active fragments, active variants) having more than 90% sequence identity to SEQ ID NO: 5 and the function of the peptide with the amino acid sequence as shown in SEQ ID NO: 5;
D. the HLA-G5 comprises: a peptide with the amino acid sequence as shown in SEQ ID NO: 6; or a derivative peptide having more than 90% sequence identity to SEQ ID NO: 6 and the function of the peptide with the amino acid sequence as shown in SEQ ID NO: 6.

In one or more embodiments, the “more than 90% sequence identity” comprises more than 91%, more than 92%, more than 93%, more than 94%, more than 95%, more than 96%, more than 97%, more than 98% or more than 99% sequence identity.

In the second aspect, the disclosure provides an immunocompatible human pluripotent stem cell, wherein it (a) does not express free B2M protein but expresses HLA-G1 and secreted HLA-G5; and (b) does not express CIITA protein. As one or more preferable embodiments of the disclosure, the human pluripotent stem cell comprises an endogenous B2M gene in the genome fusion with a polynucleotide encoding HLA-G1; comprises an exogenous nucleotide encoding a B2M-HLA-G5 fusion protein; and a deleted CIITA gene in the genome.

In one or more embodiments, the immunocompatible human pluripotent stem cell is constructed by the method for preparing immunocompatible human pluripotent stem cells in the disclosure.

In the third aspect, the disclosure provides a use of the immunocompatible human pluripotent stem cells for preparing cells suitable for transplantation by inducing differentiation; preferably, the cells suitable for transplantation are tissue or organ cells; more preferably, the tissue or organ cells comprise (but are not limited to): cardiovascular progenitor cells, cardiomyocytes, endothelial cells, smooth muscle cells, neuronal cells, hematopoietic stem cells, myeloid cells (comprising granulocytes, monocytes, macrophages, erythrocytes, platelets), lymphoid cells (comprising natural killer cells, T-cells, B-cells), retinal pigment epithelial cells, pancreatic β-cells, liver cells (comprising hepatocytes, cholangiocytes, liver endothelial cells, hepatic stellate cells, Kupffer cells, mesothelial cells), keratinocytes, skeletal muscle cells, adipocytes, osteocytes, chondrocytes, mesenchymal stem cells.

In the fourth aspect, the disclosure provides a method for preparing cells suitable for transplantation, comprising: (a) preparing immunocompatible human pluripotent stem cells by the method for preparing immunocompatible human pluripotent stem cells in the disclosure; (b) further inducing differentiation of the cells in (a) to obtain cells suitable for transplantation; preferably, the cells suitable for transplantation are tissue or organ cells; more preferably, the tissue or organ cells comprise (but are not limited to): cardiovascular progenitor cells, cardiomyocytes, endothelial cells, smooth muscle cells, neuronal cells, hematopoietic stem cells, myeloid cells (comprising granulocytes, monocytes, macrophages, erythrocytes, platelets), lymphoid cells (comprising natural killer cells, T-cells, B-cells), retinal pigment epithelial cells, pancreatic β-cells, liver cells (comprising hepatocytes, cholangiocytes, liver endothelial cells, hepatic stellate cells, Kupffer cells, mesothelial cells), keratinocytes, skeletal muscle cells, adipocytes, osteocytes, chondrocytes, mesenchymal stem cells.

In one or more embodiments, in the method for preparing cells suitable for transplantation, wherein the tissue or organ cells are cardiomyocytes, prepared using CHIR99021 and IWR-1 sequential induction; or the tissue or organ cells are endothelial cells, prepared using CHIR99021, bFGF, VEGF+BMP4 sequential induction.

In one or more embodiments, the CHIR99021, IWR-1, bFGF, VEGF or BMP4 also comprises molecules with the same functions thereof.

In one or more embodiments, the CHIR99021 has a final concentration of 6 μM, and the final concentration can fluctuate within 50%, preferably within 40%, and preferably within 30%, preferably within 20% and preferably within 10%. Preferably, the CHIR99021 is treated for 2±0.5 days.

In one or more embodiments, the IWR-1 has a final concentration of 5 μM, and the final concentration can fluctuate within 50%, preferably within 40%, and preferably within 30%, preferably within 20% and preferably within 10%. Preferably, the IWR-1 is treated for 2±0.5 days.

In one or more embodiments, the bFGF has a final concentration of 50 ng/ml, and the final concentration can fluctuate within 50%, preferably within 40%, and preferably within 30%, preferably within 20% and preferably within 10%. Preferably, the bFGF is treated for 1±0.5 days.

In one or more embodiments, the VEGF has a final concentration of 50 ng/ml, and the final concentration can fluctuate within 50%, preferably within 40%, and preferably within 30%, preferably within 20% and preferably within 10%.

In one or more embodiments, the BMP4 has a final concentration of 50 ng/ml, and the final concentration can fluctuate within 50%, preferably within 40%, and preferably within 30%, preferably within 20% and preferably within 10%.

In one or more embodiments, the VEGF+BMP4 is treated for 2±0.5 days.

In the fifth aspect, the disclosure provides a cardiomyocyte or endothelial cell, derived from immunocompatible human pluripotent stem cells prepared by the method for preparing the immunocompatible human pluripotent stem cells in the disclosure or immunocompatible human pluripotent stem cells of the disclosure.

In the sixth aspect, the disclosure provides a use of cardiomyocytes in the preparation of compositions or drugs for treating heart-related diseases or disorders; preferably, the heart-related diseases or disorders are myocardial injury, myocardial infarction, myocardial ischemia, ischemia-reperfusion injury, or other heart injuries; more preferably, the heart-related diseases or disorders is ischemia/reperfusion injury.

Other aspects of the present disclosure will be apparent to those skilled in the art based on the disclosure herein.

### DESCRIPTION OF DRAWINGS

FIG. 1. Construction and acquisition of immunocompatible cells.

FIG. 2. Construction and acquisition of immunocompatible cells. A, gRNAs were designed at the Exon2 of B2M and the Exon3 of CIITA, followed by co-transfection of the gRNA expression plasmid and Cas9 expression plasmid into hPSCs. Single clones were then picked and PCR sequencing was performed for verification. Clones with frameshift mutations are B2M and CIITA double knockout clones. B, First, B2Mm/sHLAG hPSCs were constructed (see CN113528448A), followed by co-transfection of gRNA and Cas9 plasmids. After picking single clones, PCR sequencing was performed for verification. Clones with frameshift mutations are CIITA−/−B2Mm/sHLAG-hPSCs.

FIG. 3. The expression of pluripotency marker OCT4 and SSEA4 in wt-hPSCs, DKO-hPSCs and CIITA−/− B2Mm/sHLAG-hPSCs were identified by flow cytometry. A, OCT4 expression (left) and the percentage of OCT4-positive cells (right) were analysed by flow cytometry. B, SSEA4 expression (left) and the percentage of SSEA4-positive cells (right) were analysed by flow cytometry. n=3.

FIG. 4. The expression of pluripotency marker OCT4 and SOX2 in wt-hPSCs, DKO-hPSCs and CIITA−/− B2Mm/sHLAG-hPSCs were identified by immunofluorescence. Scale bar: 20 μm.

FIG. 5. Expression of HLA molecules. A, Expression of representative HLA I molecules HLA-A/B/C was examined by flow cytometry. B, Expression of HLA-G1 was examined by flow cytometry. C, Membrane enrichment of HLA-DR was analyzed by flow cytometry, n=3. D, Expression of secreted HLA-G5 in cell supernatant was examined by Western blot. *p<0.05.

FIG. 6. Identification of differentiated endothelial cells and cardiomyocytes. A, Morphology and purity identification of differentiated endothelial cells. Purity was evaluated by flow cytometry analysis of endothelial marker CD144 expression. Scale bar=100 μm. n=3. B, Morphology and purity identification of differentiated cardiomyocytes. Purity was evaluated by flow cytometry analysis of cardiomyocyte marker cTNT expression. Scale bar=100 μm. n=3. C, Expression of sarcomeric protein α-ACTININ and gap protein Connexin-43 in differentiated cardiomyocytes was identified by immunofluorescence. Scale bar=10 μm.

FIG. 7. Evaluation of the recognition and killing effect of immune cells on cardiomyocytes derived from CIITA−/− B2Mm/sHLAG-hPSCs. Flow cytometry analysis of the effects of wt-CMs, CIITA B2Mm/sHLAG-CMs co-cultured with hPBMCs on early T-cell activation (A), T-cell proliferation (B), cytotoxicity of hPBMCs on co-cultured cardiomyocytes (C) and IFN-γ secretion by hPBMCs (D). Cardiomyocyte cytotoxicity (E) and IFN-γ secretion by NK-92 cells (F) of co-cultured NK-92 with DKO-CMs or CIITA B2Mm/sHLAG-CMs. n=5 (A); n=4 (B); n=5 (C); n=5 (D); n=6 (E); n=5 (F). *p<0.05, ***p<0.001.

FIG. 8. Evaluation of cell retention after transplantation of hPSCs-derived cardiomyocytes in humanized mice after myocardial infarction. A, Study schematic. B, Immunofluorescence image of heart sections on day 28 post-transplantation. α-ACTININ: sarcomeric protein. hu-KU80: human KU80 protein. The antibody used herein only binds to human KU80 protein and does not cross-react with mouse KU80. Scale bar=500 μm. C, Cell residency statistics (calculated as area). Female mice, n=3-7; male mice, n=3. D, Cell residency statistics (calculated by qRT-PCR), female mice, n=4-5; male mice, n=3. *p<0.05, **p<0.01, ***p<0.001.

FIG. 9. Evaluation of cardiac remuscularization in Hu-mice myocardial infarction. A, Masson staining of mouse heart sections on day 28 post-transplantation. Scale bar=1 mm. B, Statistics of heart remuscularization ratio in mice. Female mice, n=3-7; male mice, n=3. *p<0.05, **p<0.01, ***p<0.001.

FIG. 10. Other methods for constructing immune-compatible human induced pluripotent stem cells (iPSC-IC). A, Exogenous genes were inserted at a safe harbor locus (AAVS1 locus) in the genome; B, Exogenous genes were integrated into the genome using a transposon system; C, Two methods for constructing expression cassettes.

FIG. 11. Detection of exogenous gene expressions in monoclonal human iPSC-derived cardiomyocytes constructed by inserting exogenous genes into the genome using non-viral vectors. A, Flow cytometry analysis of HLA-G1 expression on the membrane of cardiomyocytes differentiated from each line. The right panel shows the statistical average fluorescence intensity; B, Western blot detection of secreted HLA-G5 in cardiomyocytes differentiated from each line; C, ELISA detection of secreted HLAG5 in cardiomyocytes differentiated from each line.

### DETAILED DESCRIPTION

In the process of applying B2Mm/sHLAG hESCs, the inventors discovered that improvements were still needed in terms of immunocompatibility. When B2Mm/sHLAG hESCs were primarily applied to cell therapy for degenerative diseases, they performed well. However, when used in other transplant environments, immune cell activation (including antigen-presenting cells) was still observed. For example, when the inventors applied these cells in cell therapy for acute injuries, such as in the infarcted heart, the inflammation level in the damaged area was high. Clinically, transplanted exogenous cells need to be implanted into the damaged or near-damaged area to exert therapeutic effects. However, damaged tissues are often accompanied by excessive activation of inflammatory responses in the diseased region, resulting in higher levels of inflammatory cytokines in the affected tissue. When transplanting hESC-derived tissue/organ cells into such environments, the immune rejection response against these cells is enhanced. After extensive experimental research and analysis of numerous molecules potentially related to these phenomena, the inventors identified targets for further modification. Based on this, the present disclosure also reveals a further modified immune-compatible cell with a broader range of application scenarios.

### TERMS

The term “pluripotent stem cells (PSCs)” refers to cells that are capable of self-renewal and proliferation while maintaining an undifferentiated state, and can, under appropriate conditions, be induced to differentiate into specialized cell types. As used herein, “pluripotent stem cells” comprise pluripotent stem cells and other types of stem cells, comprising embryonic, amniotic, or somatic stem cells. As used herein, human pluripotent stem cells (hPSCs) may be human embryonic stem cells (hESCs), isolated or obtained from human embryos within 14 days post-fertilization that have not undergone in vivo development. Exemplary human stem cell lines comprise H9 human pluripotent stem cell line, for example, commercially available H9hESC cell line. Additional exemplary stem cell lines comprise those available through National Institutes of Health Human Embryonic Stem Cell Registry and Howard Hughes Medical Institute HUES collection (e.g., Cowan, C. A., et al., New England J. Med. 350:13. (2004)).

As used herein, “pluripotent stem cells” have the potential to differentiate into any of the following three germ layers: endoderm (e.g., gastric junction, gastrointestinal tract, lung, etc.), mesoderm (e.g., muscle, bone, blood, urogenital tissue, etc.) or ectoderm (such as epidermal tissue and nervous system tissue). The term “pluripotent stem cells” used herein also comprises “induced pluripotent stem cells”, “iPS”, “iPSC” or “iPSCs”, a type of pluripotent stem cells derived from non-pluripotent cells. Examples of parental cells comprise somatic cells that have been reprogrammed by various means to induce a pluripotent, undifferentiated phenotype. Such “iPS”, “iPSC” or “iPSCs” cells can be generated by inducing the expression of certain regulatory genes or by the exogenous application of certain proteins. Methods of inducing iPS cells are known in the art. As used herein, “hiPSC”, “hiPSCs”, “hPSC” or “hPSCs” are human induced pluripotent stem cells, and “miPSC”, “miPSCs”, “mPSC” or “mPSCs” are murine induced pluripotent stem cells.

The term “pluripotent stem cell characteristics” refers to cellular characteristics that distinguish pluripotent stem cells from other cells. The ability to produce progeny that can differentiate under appropriate conditions into cell types that collectively display characteristics associated with cell lineages from all three germinal layers (endoderm, mesoderm, and ectoderm) is a pluripotent stem cell characteristic. The expression or non-expression of certain combinations of molecular markers is also characteristic of pluripotent stem cells. For example, human pluripotent stem cells express at least several, and in some embodiments all markers from the following non-limiting list: SSEA-3, SSEA-4, TRA-1-60, TRA-1-81, TRA-2-49/6E, ALP, Sox2, E-cadherin, UTF-1, Oct4, Rex1 and Nanog. Cell morphologies associated with pluripotent stem cells are also characteristic of pluripotent stem cells. As described herein, cells do not need pluripotency to be reprogrammed into endodermal progenitors and/or hepatocytes.

The term “multipotent” or “multipotent cell” refers to a cell type that can produce a limited number of other specific cell types. For example, induced pluripotent cells can form endodermal cells. Additionally, multipotent hematopoietic stem cells can differentiate into several types of blood cells, comprising lymphocytes, monocytes, neutrophils, etc. The term “oligopotent” refers to the ability of adult stem cells to differentiate into only a few different cell types. For example, lymphoid or bone marrow stem cells can form cells from the lymphoid or bone marrow lineage, respectively. The term “unipotent” refers to the ability of a cell to form a single cell type. For example, spermatogonial stem cells can only form sperm cells.

The term “non-pluripotent cells” refers to mammalian cells that are not pluripotent. Examples of such cells comprise differentiated cells and progenitor cells. Examples of differentiated cells comprise but are not limited to cells from tissues selected from bone marrow, skin, skeletal muscle, adipose tissue, and peripheral blood. Exemplary cell types comprise but are not limited to fibroblasts, hepatocytes, myocytes, neurons, osteoblasts, osteoclasts, and T-cells. Starting cells for generating induced pluripotent cells, endodermal progenitor cells and hepatocytes can be non-pluripotent cells. Differentiated cells comprise but are not limited to pluripotent cells, oligopotent cells, unipotent cells, progenitor cells and terminally differentiated cells. In certain embodiments, cells with lower potency are considered “differentiated” relative to cells with higher potency.

The term “somatic cells” refers to cells that form an organism. Somatic cells comprise cells that form organs, skin, blood, bones, and connective tissues of the organism, but exclude reproductive cells. Cells may be derived from, for example, humans or non-human mammals. Exemplary non-human mammals comprise but are not limited to mice, rats, cats, dogs, rabbits, guinea pigs, hamsters, sheep, pigs, horses, cows and non-human primates. In some embodiments, cells are derived from adult or non-human mammals. In some embodiments, cells are derived from neonates, adult or non-human mammals.

As used herein, the term “subject” or “patient” refers to any animal, such as domesticated animals, zoo animals, or humans. A “subject” or “patient” can be a mammal, such as a dog, cat, bird, or livestock, comprising a human. Specific examples of “subjects” and “patients” comprise but are not limited to individuals with diseases or conditions related to the liver, heart, lungs, kidneys, pancreas, brain, nervous tissue, blood, bones, bone marrow, etc. (particularly humans). Mammalian cells may be derived from human or non-human mammals. Exemplary non-human mammals comprise but are not limited to mice, rats, cats, dogs, rabbits, guinea pigs, hamsters, sheep, pigs, horses, cows and non-human primates (for example, chimpanzees, macaques and apes).

The term “HLA” or “human leukocyte antigen” complex is a complex of genes encoding human major histocompatibility complex (MHC) proteins. These cell surface proteins that make up the HLA complex are responsible for regulating the immune response to antigens. In humans, there are two types of HLA: Class I and Class II, “HLA I” and “HLA II”. HLA I consists of at least three proteins, HLA-A, HLA-B and HLA-C, and these proteins present peptides from inside the cell. HLA II comprises at least five proteins: HLA-DP, HLA-DM, HLA-DOB, HLA-DQ and HLA-DR, wherein these proteins present extracellular antigens to T lymphocytes. It should be understood that the use of “MHC” or “HLA” does not imply limitation, as it depends on whether the gene is from humans (HLA) or mice (MHC). Therefore, when referring to mammalian cells, these terms can be used interchangeably herein.

The term “gene knockout” refers to a process of inactivating a specific gene in its host cell, resulting in the failure to produce the target protein or an inactive form thereof. As understood by those skilled in the art and described further below, this can be achieved in a number of different ways, including removing all or part of the nucleic acid sequence from the gene, or interrupting the sequence with other sequences, removing or altering regulatory components (e.g. promoters) to prevent genes from being transcribed, altering the reading frame, blocking translation by binding to mRNA, or modifying the regulatory components of the nucleic acid, etc. For example, the entire or part of the coding region of the target gene can be removed or replaced with a “nonsense” sequence, entire or partial regulatory sequences (e.g., promoters) can be removed or replaced, or translation initiation sequences can be removed or replaced. Typically, knockout is performed at the genomic DNA level, so that the progeny of the cells also permanently carries the knockout.

The term “gene knock-in/introduction” refers to a process of adding genetic functions to the host cell. This results in an increase in the level of encoded proteins. As understood by those skilled in the art, this can be achieved by several means, comprising adding one or more additional copies of a gene into the host cell or modifying the regulatory components of the endogenous gene to increase protein expression. This can be achieved by modifying the promoter, adding different promoters, adding enhancers, or modifying other gene expression sequences. Typically, the knock-in leads to the integration of transgenic additional copies into the host cell.

The term “β-2 microglobulin” or “β2M” or “B2M” protein may refer to a human β2M protein having an amino acid sequence shown in SEQ ID NO: 2 below, or having more than 90% sequence identity and similar biological activity to the amino acid sequence shown in SEQ ID NO: 2. The term “CIITA” protein may refer to a human CIITA protein having an amino acid sequence shown in SEQ ID NO: 5 below, or having more than 90% sequence identity and similar biological activity to the amino acid sequence shown in SEQ ID NO: 5. In the context of cells, “wild type” refers to cells found in nature. However, in the context of pluripotent stem cells, as used herein, it also means iPSCs that may comprise nucleic acid changes that lead to pluripotency but do not undergo the gene editing procedures of the disclosure to achieve low immunogenicity.

The term “allogeneic” refers to the genetic differences between the host organism and the transplanted cells, wherein the immune response is generated.

“B2M^{-/-}” used herein means that diploid cells have inactive B2M genes in both chromosomes. This can be done in various ways. Similarly, “CIITA^{-/-}” used herein refers to diploid cells with inactive CIITA genes in both chromosomes. This can be done in various ways, as described herein.

As used herein, “m/sHLAG” is an abbreviation for membrane (m) localized HLA-G1 and soluble (s) HLA-G5.

As used herein, “B2Mm/sHLAG hPSCs” cell lines are cell lines that overexpress both membrane-localized HLA-G1 and soluble HLA-G5.

As used herein, the “expression” comprises “overexpression” and “recombinant expression”. The “overexpression”, for example, means that the expression level is significantly increased compared to the expression level in the wild type, such as increased to more than 1.2, 1.5, 2, 3, 5, 8, 10, 15, 20, 30, 50 times or higher of the wild type.

As used herein, “no expression” is a relative term, and also encompasses “low expression” and “extremely low expression”. For example, compared with the expression level in the wild type, the expression of the modified cells is reduced to below 15%, below 10%, below 8%, below 5%, below 3%, below 2%, below 1% or less than that of the wild type.

As used herein, the term “comprise” means that various components can be used together in the mixture or composition of the present disclosure. Therefore, the term “mainly consist of . . . ” and “consist of . . . ” is included in the term “comprise”. As used herein, the term “effective amount” or “effective dose” refers to an amount that is functional or active for subjects and is acceptable for administration to humans and/or animals.

As used herein, “pharmaceutically acceptable” ingredients refer to substances suitable for use in subjects without undue adverse side effects (such as toxicity, irritation, and allergic reactions), i.e. with reasonable benefit/risk ratio. The term “pharmaceutically acceptable carrier” refers to a carrier for the administration of a therapeutic agent, comprising various excipients and diluents.

Every maximum numerical limitation given throughout the disclosure comprises every lower numerical limitation as if such lower numerical limitations were expressly written herein. Every minimum numerical limitation given throughout the disclosure comprises every higher numerical limitation as if such higher numerical limitation were expressly written herein. Every numerical range given throughout the disclosure comprises every narrower numerical range that falls within such broader numerical range, as if such narrower numerical ranges were expressly written herein.

### Cells of the Disclosure and the Use Thereof

The disclosure provides a human pluripotent stem cell that avoids host immune responses due to the genetic manipulation of the disclosure. Specifically, the genome of the human pluripotent stem cell has been modified to: not express free B2M protein, not express CIITA protein, but express (exogenous) HLA-G1 and secreted HLA-G5. Those skilled in the art can understand that human pluripotent stem cells constructed by the method of the disclosure are also within the protective scope of the disclosure.

The human pluripotent stem cells allow the derivation of “ready-to-use” cell products for the generation of specific tissues and organs. The derivatives of these human pluripotent stem cells used in human patients provide significant benefits, comprising the ability to avoid long-term adjuvant immunosuppressive therapies and drugs typically seen in transplantation. For example, cardiomyocytes derived from these cells can evade recognition and killing by human immune cells, exhibiting better retention effects in cardiac ischemia/reperfusion injury models. It can also significantly reduce costs, as cell therapy can be applied without the need for individual treatment for each patient. Therefore, the human pluripotent stem cells are immunocompatible (avoiding host immune responses), making them suitable for a broader patient population and serving as a universal cell source for generating widely accepted derivatives.

The disclosure provides a human pluripotent stem cell that avoids host immune responses due to several genetic manipulations of the disclosure. The cells lack major immune antigens that trigger immune responses and are engineered to escape recognition and killing by immune cells.

Specifically, the human pluripotent stem cells of the disclosure do not express free B2M protein and CIITA protein, but, as a preferred embodiment, they can successfully express secreted B2M-HLA-G5 fusion protein. In some embodiments, the human pluripotent stem cells of the disclosure are achieved by integrating recombinant nucleic acids into the genome of the human pluripotent stem cells.

Exemplary methods for genetic manipulation comprise homologous recombination, knock-in, ZFNs (zinc finger nucleases), TALENs (transcription activator-like effector nucleases), CRISPR (clustered regularly interspaced short palindromic repeats)/Cas9 and other site-specific nuclease technologies. There are a number of CRISPR/Cas9 based technologies, see for example Doudna and Charpentier, Science doi: 10.1126/science.1258096, wherein it is incorporated herein by reference. These techniques enable double-stranded DNA breaks at desired locus sites. These controlled double-strand breaks promote homologous recombination at specific loci. The process focuses on targeting specific sequences in nucleic acid molecules, such as chromosomes, using nucleases that recognize and bind to the sequence, inducing double-strand breaks in nucleic acid molecules. The double-strand breaks are repaired through error-prone non-homologous end joining (NHEJ) or homologous recombination (HR).

As understood by those skilled in the art, many different techniques can be used to modify the pluripotent stem cells of the disclosure, as well as to render human pluripotent stem cells deficient in major immune antigens that trigger an immune response and engineered to evade recognition and killing by immune cells as described herein.

Typically, these techniques can be used individually or in combination. For example, in the construction of human pluripotent stem cells, CRISPR can be used to reduce the expression of active B2M and/or CIITA proteins in modified cells, and virus (such as lentivirus) can be used for stable gene transduction. Additionally, as understood by those skilled in the art, while in one embodiment, sequentially B2M is knockout by CRISPR/Cas9, followed by knockout of CIITA via CRISPR/Cas9, these genes can be manipulated using different methods or in a different order. As a preferred embodiment, immunocompatible human pluripotent stem cells were constructed by lentiviral infection. In this process, lentiviral vectors usually require steps such as plasmid extraction, virus packaging and quality control, and are randomly integrated into the genome. As a preferred embodiment, a non-viral plasmid transfection method is used, with targeted knock-in or transposon systems (e.g., Piggybac, Sleeping Beauty, etc.) to integrate the target gene into the genome of the target cells. Compared to the lentivirus system, this method offers advantages in terms of production, preparation process management and safety.

In the disclosure, multiple methods can be used to screen for single clone cell lines, such as drug screening, PCR screening, flow cytometry screening, ELISA screening, Western blot, etc.

For all of these methods, well-known recombinant technologies are used to produce the recombinant nucleic acids described herein. In certain embodiments, recombinant nucleic acids (encoding the desired polypeptides, such as the B2M-HLA-G1 fusion protein or CIITA protein) can be operably linked with one or more regulatory nucleotide sequences in the expression construct. The regulatory nucleotide sequences are typically suitable for the host cell and the subject to be treated. Various types of suitable expression vectors and regulatory sequences for multiple host cells are known in the art. Typically, one or more regulatory nucleotide sequences may comprise but are not limited to promoter sequences, leader sequences or signal sequences, ribosome binding sites, transcriptional initiation and termination sequences, translational initiation and termination sequences, as well as enhancer or activator sequences. Constitutive or inducible promoters known in the art are also contemplated. The promoter may be a naturally occurring promoter, or a hybrid promoter combining elements of more than one promoter. The expression construct may be present on an episome (e.g., a plasmid) in cells, or the expression construct may be inserted into the chromosome. In a specific embodiment, the expression vector comprises a selectable marker gene to allow selection of transformed host cells. Some embodiments comprise expression vectors comprising a nucleotide sequence encoding a variant polypeptide operably linked to at least one regulatory sequence. Regulatory sequences as used herein include promoters, enhancers and other expression control elements. In certain embodiments, expression vectors are designed for selection of the host cell to be transformed, the specific variant polypeptide desired to be expressed, the copy number of the vector, the ability to control the copy number, or any other protein encoded by the vector such as an antibiotic marker expression.

The present disclosure also provides cardiomyocytes derived from the human pluripotent stem cells and then transplanted into patients in need, thereby avoiding recognition and killing by human immune cells. The disclosure also provides a use of the derived cardiomyocytes in the preparation of compositions or drugs for treating heart-related diseases or disorders.

As understood by those skilled in the art, human pluripotent stem cells allow the derivation of “ready-to-use” cell products for the generation of specific tissues and organs. The derivatives of these human pluripotent stem cells used in human patients provide significant benefits, comprising the ability to avoid long-term adjuvant immunosuppressive therapies and drugs typically seen in transplantation. For example, cardiomyocytes derived from these cells can evade recognition and killing by human immune cells, exhibiting better retention effects in cardiac ischemia/reperfusion injury models. It can also significantly reduce costs, as cell therapy can be applied without the need for individual treatment for each patient. Therefore, the human pluripotent stem cells are immunocompatible, making them suitable for a broader patient population and serving as a universal cell source for generating widely accepted derivatives.

The disclosure also provides a human pluripotent stem cell with ideal immunocompatibility (on avoiding host immune responses) due to several genetic manipulations of the disclosure. These cells lack major immune antigens that trigger immune responses and are engineered to be more immunocompatible (on avoiding host immune responses). Those skilled in the art can understand that human pluripotent stem cells constructed by the method of the disclosure are also within the protective scope of the disclosure.

The present disclosure provides cardiomyocytes derived from the human pluripotent stem cells and then transplanted into patients in need, thereby avoiding recognition and killing by human immune cells. The disclosure also provides a use of the derived cardiomyocytes in the preparation of compositions or drugs for treating heart-related diseases or disorders. Preferably, the heart-related diseases or disorders are myocardial injury, myocardial infarction, myocardial ischemia, ischemia-reperfusion injury, or other heart injuries; more preferably, the heart-related diseases or disorders are ischemia/reperfusion injury. In other words, the disclosure provides a use of the derived cardiomyocytes in the preparation of compositions or drugs for promoting myocardial repair after myocardial infarction, improving cardiac function after myocardial infarction and protecting against myocardial ischemic injury.

In a preferable embodiment, the heart-related diseases or disorders are myocardial injury, myocardial infarction, myocardial ischemia, ischemia-reperfusion injury or other heart injuries; more preferably, the heart-related diseases or disorders are ischemia/reperfusion injury. In other words, the disclosure provides a use of the derived cardiomyocytes in the preparation of compositions or drugs for promoting myocardial repair after myocardial infarction, improving cardiac function after myocardial infarction and protecting against myocardial ischemic injury.

From another perspective, the present disclosure provides cardiomyocytes derived from the human pluripotent stem cells and then transplanted into patients in need, thereby avoiding recognition and killing by human immune cells. The disclosure also provides a use of the derived cardiomyocytes in the preparation of compositions or drugs for treating heart-related diseases or disorders. As a preferable embodiment, the heart-related diseases or disorders are myocardial injury, myocardial infarction, myocardial ischemia, ischemia-reperfusion injury or other heart injuries. As a more preferable embodiment, the heart-related diseases or disorders are ischemia/reperfusion injury. In other words, the disclosure provides a use of cardiomyocytes derived from the human pluripotent stem cells in the preparation of compositions or drugs for promoting myocardial repair after myocardial infarction, improving cardiac function after myocardial infarction and protecting against myocardial ischemic injury.

From a therapeutic perspective, it can also be said that the disclosure provides a use of cardiomyocytes derived from the human pluripotent stem cells for treating heart-related diseases or disorders, such as myocardial injury, myocardial infarction, myocardial ischemia, ischemia-reperfusion injury and other related diseases or disorders. As a more preferable embodiment, the disclosure provides a use of cardiomyocytes derived from the human pluripotent stem cells for promoting myocardial repair after myocardial infarction, improving cardiac function after myocardial infarction and protecting against myocardial ischemic injury.

The present disclosure also provides a composition, wherein it comprises an effective amount of the derived cardiomyocytes and a pharmaceutically acceptable carrier. Such carriers comprise (but are not limited to): saline, buffer, glucose, water, glycerol, ethanol, and combinations thereof. Usually, the pharmaceutical formulation should match the route of administration. The pharmaceutical composition of the present disclosure can be prepared in the form of an injection, for example, using a saline solution or a water solution containing glucose and other excipients through conventional methods. The pharmaceutical composition should be manufactured under sterile conditions. The amount of active ingredient administered is a therapeutically effective amount. The composition of the present disclosure can be used directly for promoting myocardial repair after myocardial infarction, improving cardiac function after myocardial infarction and protecting against myocardial ischemic injury. In addition, it can also be used in combination with other active agents or adjuvants at the same time.

Generally, these materials can be formulated in a non-toxic, inert medium with pharmaceutically acceptable aqueous carrier, wherein the pH is usually about 5-8, preferably the pH is about 6-8.

The effective amount of the derived cardiomyocytes in the present disclosure may vary with the mode of administration, the severity of the disease to be treated, and the like. Selection of preferred effective amount can be determined by those skilled in the art based on various factors (e.g. through clinical trials). Such factors comprise but are not limited to: pharmacokinetic parameters (such as bioavailability, metabolism, half-life, and so on), the severity of the disease to be treated, weight, immune status of patients, the form of administration, and so on. The route of administrating the derived cardiomyocytes of the present disclosure is not particularly limited. It may be systemic or local. For example, the derived cardiomyocytes of the present disclosure can be administered by local tissue injection, preferably myocardial injection. In addition, other methods of injection are also possible, such as, but not limited to, intraperitoneal injection, intravenous injection, oral administration, subcutaneous injection, intrathecal injection, intradermal injection and other methods of administration to the subject.

### METHODS OF THE DISCLOSURE

The present disclosure provides a method for constructing human pluripotent stem cells, comprising: modifying the human pluripotent stem cells to: not express free B2M protein, express HLA-G1 and secreted HLA-G5; and not express CIITA protein.

In the constructing method provided by the present disclosure, an encoding gene of HLA-G1 fragment may comprise an encoding sequence of the heavy chain reading frame of HLA-G1, and the HLA-G1 fragment may comprise:
a) A polypeptide fragment with an amino acid sequence as shown in SEQ ID NO: 1; or,
b) A polypeptide fragment with an amino acid sequence of more than 90% sequence identity with SEQ ID NO: 1 and the function of the polypeptide fragment in a).

The amino acid sequence of heavy chain of HLA-G1 (HLA-G1 fragment) is as follows:

(SEQ ID NO: 1)
GSHSMRYFSAAVSRPGRGEPRFIAMGYVDDTQFVRFDSDSACPRMEPR
APWVEQEGPEYWEEETRNTKAHAQTDRMNLQTLRGYYNQSEASSHTLQ
WMIGCDLGSDGRLLRGYEQYAYDGKDYLALNEDLRSWTAADTAAQISK
RKCEAANVAEQRRAYLEGTCVEWLHRYLENGKEMLQRADPPKTHVTHH
PVFDYEATLRCWALGFYPAEIILTWQRDGEDQTQDVELVETRPAGDGT
FQKWAAVVVPSGEEQRYTCHVQHEGLPEPLMLRWKQSSLPTIPIMGIV
AGLVVLAAVVTGAAVAAVLWRKKSSD*

Specifically, the polypeptide fragment in b) specifically refers to: a polypeptide fragment with one or more (specifically 1-50, 1-30, 1-20, 1-10, 1-5, 1-3, 1, 2 or 3) amino acid substitution, deletion or addition of the amino acid sequence shown in SEQ ID NO: 1, or a polypeptide fragment with one or more (specifically 1-50, 1-30, 1-20, 1-10, 1-5, 1-3, 1, 2 or 3) amino acids added at the N-terminus and/or C-terminus and the function of the polypeptide fragment with the amino acid sequence shown in SEQ ID NO: 1.

For example, the HLA-G1 fragment usually has a complete α-heavy chain structure. unlike classical HLA I molecules, it mainly exhibits immunosuppressive functions (for example, binding to inhibitory receptors, thereby regulating immune responses mediated by B cells, T cells, NK cells, and APC cells, wherein the inhibitory receptors mainly comprise ILT2/CD85j/LILRB1, ILT4/CD85d/LILRB2, and KIR2DL4/CD158d, etc.). The amino acid sequence in b) may have more than 90%, more than 93%, more than 95%, more than 97% or more than 99% sequence identity with SEQ ID NO: 1. The HLA-G1 fragment usually originated from human.

In the constructing method provided by the present disclosure, the first B2M fragment may comprise: c) a polypeptide fragment with an amino acid sequence as shown in SEQ ID NO: 2; or d) a polypeptide fragment with an amino acid sequence of more than 90% sequence identity with SEQ ID NO: 1 and the function of the polypeptide fragment in c).

The amino acid sequence of endogenous B2M protein (first B2M fragment) is as follows:

(SEQ ID NO: 2)
MSRSVALAVLALLSLSGLEAIQRTPKIQVYSRHPAENGKSNFLNCYVS
GFHPSDIEVDLLKNGERIEKVEHSDLSFSKDWSFYLLYYTEFTPTEKD
EYACRVNHVTLSQPKIVKWDRDM.

Specifically, the polypeptide fragment in d) specifically refers to: a polypeptide fragment with one or more (specifically 1-50, 1-30, 1-20, 1-10, 1-5, 1-3, 1, 2 or 3) amino acid substitution, deletion or addition of the amino acid sequence shown in SEQ ID NO: 2, or a polypeptide fragment with one or more (specifically 1-50, 1-30, 1-20, 1-10, 1-5, 1-3, 1, 2 or 3) amino acids added at the N-terminus and/or C-terminus and the function of the polypeptide fragment with the amino acid sequence shown in SEQ ID NO: 2. For example, the first B2M fragment usually has a β-pleated sheet structure and it mainly has the function of binding to the major histocompatibility complex class I heavy chain through non-covalent bonds. The amino acid sequence in d) may have more than 90%, more than 93%, more than 95%, more than 97% or more than 99% sequence identity with SEQ ID NO: 2. The first B2M fragment usually originated from human.

In the constructing method provided by the present disclosure, the B2M-HLA-G1 fusion protein may also comprise a first flexible linker peptide, and the first flexible linker peptide is usually located between the HLA-G1 fragment and the first B2M fragment. The first flexible linker peptide is typically a flexible polypeptide of appropriate length consisting of glycine (G), serine (S), and/or alanine (A), allowing adjacent protein domains to move relative to each other freely. For example, the amino acid sequence of the first flexible linker peptide can comprise sequences such as (GS)ₙ, (GGS)ₙ, (GGSG)ₙ, (GGGS)ₙA, (GGGGS)ₙA, (GGGGA)ₙA, (GGGGG)ₙA, wherein n is an integer selected from 1 to 10. In some specific embodiments of the present disclosure, the length of the amino acid sequence of the first flexible linker peptide may be 5-26. In some specific embodiments of the present disclosure, the first flexible linker peptide may comprise a polypeptide fragment with an amino acid sequence as shown in SEQ ID NO: 3. In other specific embodiments of the present disclosure, the B2M-HLA-G1 fusion protein comprises, from the N-terminus to the C-terminus, the first B2M fragment and the HLA-G1 fragment, wherein the B2M-HLA-G1 fusion protein comprises a polypeptide fragment with the amino acid sequence shown in SEQ ID NO: 4.

The amino acid sequence of the flexible (G₄S)₄ linker peptide (the first flexible linker peptide) is as follows:

(SEQ ID NO: 3)
GlyGlyGlyGlySerGlyGlyGlyGlySerGlyGlyGlyGlySerGly
GlyGlyGlySer

The amino acid sequence of B2M-HLA-G1 fusion protein is as follows:

(SEQ ID NO: 4)
MSRSVALAVLALLSLSGLEAIQRTPKIQVYSRHPAENGKSNFLNCYVS
GFHPSDIEVDLLKNGERIEKVEHSDLSFSKDWSFYLLYYTEFTPTEKD
EYACRVNHVTLSQPKIVKWDRDMGGGGSGGGGSGGGGSGGGGSGSHSM
RYFSAAVSRPGRGEPRFIAMGYVDDTQFVRFDSDSACPRMEPRAPWVE
QEGPEYWEEETRNTKAHAQTDRMNLQTLRGYYNQSEASSHTLQWMIGC
DLGSDGRLLRGYEQYAYDGKDYLALNEDLRSWTAADTAAQISKRKCEA
ANVAEQRRAYLEGTCVEWLHRYLENGKEMLQRADPPKTHVTHHPVFDY
EATLRCWALGFYPAEIILTWQRDGEDQTQDVELVETRPAGDGTFQKWA
AVVVPSGEEQRYTCHVQHEGLPEPLMLRWKQSSLPTIPIMGIVAGLVV
LAAVVTGAAVAAVLWRKKSSD*

The nucleic acid sequence encoding B2M-HLA-G1 fusion protein is as follows, wherein exon DNA sequence of the endogenous B2M gene is bolded and the intron DNA sequence is underlined:

(SEQ ID NO: 9)
atgtctcgctccgtggccttagctgtgctcgcgctactctctctttctgg
cctggaggctatccagcgtgagtctctcctaccctcccgctctggtcctt
cctctcccgctctgcaccctctgtggccctcgctgtgctctctcgctccg
tgacttcccttctccaagttctccttggtggcccgccgtggggctagtcc
agggctggatctcggggaagcggcggggtggcctgggagtggggaagggg
gtgcgcacccgggacgcgcgctacttgcccctttcggggggagcagggga
gacctttggcctacggcgacgggagggtcgggacaaagtttagggcgtcg
ataagcgtcagagcgccgaggttgggggagggtttctcttccgctctttc
gcggggcctctggctcccccagcgcagctggagtgggggacgggtaggct
cgtcccaaaggcgcggcgctgaggtttgtgaacgcgtggaggggcgcttg
gggtctgggggaggcgtcgcccgggtaagcctgtctgctgcggctctgct
tcccttagactggagagctgtggacttcgtctaggcgcccgctaagttcg
catgtcctagcacctctgggtctatgtggggccacaccgtggggaggaaa
cagcacgcg660acgtttgtagaatgcttggctgtgatacaaagcggttt
cgaataattaacttatttgttcccatcacatgtcacttttaaaaaattat
aagaactacccgttattgacatctttctgtgtgccaaggactttatgtgc
tttgcgtcatttaattttgaaaacagttatcttccgccatagataactac
tatggttatcttctgcctctcacagatgaagaaactaaggcaccgagatt
ttaagaaacttaattacacaggggataaatggcagcaatcgagattgaag
tcaagcctaaccagggcttttgcgggagcgcatgccttttggctgtaatt
cgtgcatttttttttaagaaaaacgcctgccttctgcgtgagattctcca
gagcaaactgggcggcatgggccctgtggtcttttcgtacagagggcttc
ctctttggctctttgcctggttgtttccaagatgtactgtgcctcttact
ttcggttttgaaaacatgagggggttgggcgtggtagcttacgcctgtaa
tcccagcacttagggaggccgaggcgggaggatggcttgaggtccgtagt
tgagaccagcctggccaacatggtgaagcctggtctctacaaaaaataat
aacaaaaattagccgggtgtggtggctcgtgcctgtggtcccagctgctc
cggtggctgaggcgggaggatctcttgagcttaggcttttgagctatcat
ggcgccagtgcactccagcgtgggcaacagagcgagaccctgtctctcaa
aaaagaaaaaaaaaaaaaaagaaagagaaaagaaaagaaagaaagaagtg
aaggtttgtcagtcaggggagctgtaaaaccattaataaagataatccaa
gatggttaccaagactgttgaggacgccagagatcttgagcactttctaa
gtacctggcaatacactaagcgcgctcaccttttcctctggcaaaacatg
atcgaaagcagaatgttttgatcatgagaaaattgcatttaatttgaata
caatttatttacaacataaaggataatgtatatatcaccaccattactgg
tatttgctggttatgttagatgtcattttaaaaaataacaatctgatatt
taaaaaaaaatcttattttgaaaatttccaaagtaatacatgccatgcat
agaccatttctggaagataccacaagaaacatgtaatgatgattgcctct
gaaggtctattttcctcctctgacctgtgtgtgggttttgtttttgtttt
actgtgggcataaattaatttttcagttaagttttggaagcttaaataac
tctccaaaagtcataaagccagtaactggttgagcccaaattcaaaccca
gcctgtctgatacttgtcctcttcttagaaaagattacagtgatgctctc
acaaaatcttgccgccttccctcaaacagagagttccaggcaggatgaat
ctgtgctctgatccctgaggcatttaatatgttcttattattagaagctc
agatgcaaagagctctcttagcttttaatgttatgaaaaaaatcaggtct
tcattagattccccaatccacctcttgatggggctagtagcctttcctta
atgatagggtgtttctagagagatatatctggtcaaggtggcctggtact
cctccttctccccacagcctcccagacaaggaggagtagctgccttttag
tgatcatgtaccctgaatataagtgtatttaaaagaattttatacacata
tatttagtgtcaatctgtatatttagtagcactaacacttctcttcattt
tcaatgaaaaatatagagtttataatattttcttcccacttccccatgga
tggtctagtcatgcctctcattttggaaagtactgtttctgaaacattag
gcaatatattcccaacctggctagtttacagcaatcacctgtggatgcta
attaaaacgcaaatcccactgtcacatgcattactccatttgatcataat
ggaaagtatgttctgtcccatttgccatagtcctcacctatccctgttgt
attttatcgggtccaactcaaccatttaaggtatttgccagctcttgtat
gcatttaggttttgtttctttgttttttagctcatgaaattaggtacaaa
gtcagagaggggtctggcatataaaacctcagcagaaataaagaggtttt
gttgtttggtaagaacataccttgggttggttgggcacggtggctcgtgc
ctgtaatcccaacactttgggaggccaaggcaggctgatcacttgaagtt
gggagttcaagaccagcctggccaacatggtgaaatcccgtctctactga
aaatacaaaaattaaccaggcatggtggtgtgtgcctgtagtcccaggaa
tcacttgaacccaggaggcggaggttgcagtgagctgagatctcaccact
gcacactgcactccagcctgggcaatggaatgagattccatcccaaaaaa
aaaaaaataaaaaaataaagaacataccttgggttgatccacttaggaac
ctcagataataacatctgccacgtatagagcaattgctatgtcccaggca
ctctactagacacttcatacagtttagaaaatcagatgggtgtagatcaa
ggcaggagcaggaaccaaaaagaaaggcataaacataagaaaaaaaatgg
aaggggtggaaacagagtacaataacatgagtaatttgatgggggctatt
atgaactgagaaatgaactttgaaaagtatcttggggccaaatcatgtag
actcttgagtgatgtgttaaggaatgctatgagtgctgagagggcatcag
aagtccttgagagcctccagagaaaggctcttaaaaatgcagcgcaatct
ccagtgacagaagatactgctagaaatctgctagaaaaaaaacaaaaaag
gcatgtatagaggaattatgagggaaagataccaagtcacggtttattct
tcaaaatggaggtggcttgttgggaaggtggaagctcatttggccagagt
ggaaatggaattgggagaaatcgatgaccaaatgtaaacacttggtgcct
gatatagcttgacaccaagttagccccaagtgaaataccctggcaatatt
aatgtgtcttttcccgatattcctcaggtactccaaagattcaggtttac
tcacgtcatccagcagagaatggaaagtcaaatttcctgaattgctatgt
gtctgggtttcatccatccgacattgaagttgacttactgaagaatggag
agagaattgaaaaagtggagcattcagacttgtctttcagcaaggactgg
tctttctatctcttgtactacactgaattcacccccactgaaaaagatga
gtatgcctgccgtgtgaaccatgtgactttgtcacagcccaagatagtta
agtggggtaagtcttacattcttttgtaagctgctgaaagttgtgtatga
gtagtcatatcataaagctgctttgatataaaaaaggtctatggccatac
taccctgaatgagtcccatcccatctgatataaacaatctgcatattggg
attgtcagggaatgttcttaaagatcagattagtggcacctgctgagata
ctgatgcacagcatggtttctgaaccagtagtttccctgcagttgagcag
ggagcagcagcagcacttgcacaaatacatatacactcttaacacttctt
acctactggcttcctctagcttttgtggcagcttcaggtatatttagcac
tgaacgaacatctcaagaaggtataggcctttgtttgtaagtcctgctgt
cctagcatcctataatcctggacttctccagtactttctggctggattgg
tatctgaggctagtaggaagggcttgttcctgctgggtagctctaaacaa
tgtattcatgggtaggaacagcagcctattctgccagccttatttctaac
cattttagacatttgttagtacatggtattttaaaagtaaaacttaatgt
cttccttttttttctccactgtctttttcatagatcgagacatgGGTGGA
GGTGGAAGTGGTGGAGGTGGAAGTGGTGGAGGTGGaagtGGtggaggtgg
aagtggCTCCCACTCCATGAGGTATttcagcgccgccgtgtcccggcccg
gccgcggggagccccgcttcatcgccatgggctacgtggacgacacgcag
ttcgtgcggttcgacagcgactcggcgtgtccgaggatggagccgcgggc
gccgtgggtggagcaggaggggccggagtattgggaagaggagacacgga
acaccaaggcccacgcacagactgacagaatgaacctgcagaccctgcgc
ggctactacaaccagagcgaggccagttctcacaccctccagtggatgat
tggctgcgacctggggtccgacggacgcctcctccgcgggtatgaacagt
atgcctacgatggcaaggattacctcgccctgaacgaggacctgcgctcc
tggaccgcagcggacactgcggctcagatctccaagcgcaagtgtgaggc
ggccaatgtggctgaacaaaggagagcctacctggagggcacgtgcgtgg
agtggctccacagatacctggagaacgggaaggagatgctgcagcgcgcg
gacccccccaagacacacgtgacccaccaccctgtctttgactatgaggc
caccctgaggtgctgggccctgggcttctaccctgcggagatcatactga
cctggcagcgggatggggaggaccagacccaggacgtggagctcgtggag
accaggcctgcaggggatggaaccttccagaagtgggcagctgtggtggt
gccttctggagaggagcagagatacacgtgccatgtgcagcatgaggggc
tgccggagcccctcatgctgagatggaagcagtcttccctgcccaccatc
cccatcatgggtatcgttgctggcctggttgtccttgcagctgtagtcac
tggagctgcggtcgctgctgtgctgtGGAGAAAGAAGAGCTCAGATTGA

In the constructing method provided by the present disclosure, the method for integrating an exogenous nucleic acid encoding the B2M-HLA-G1 fusion protein into the genome of human pluripotent stem cells specifically comprises: fusing the gene encoding the HLA-G1 fragment with the endogenous B2M gene in human pluripotent stem cells. In some specific examples of the present disclosure, the stop codon in exon 3 of the endogenous B2M gene can be replaced by the gene encoding the HLA-G1 fragment.

In the constructing method provided by the present disclosure, the CIITA fragment may comprise: e) a polypeptide fragment with an amino acid sequence as shown in SEQ ID NO: 5; or f) a polypeptide fragment with an amino acid sequence of more than 90% sequence identity with SEQ ID NO: 5 and the function of the polypeptide fragment in e).

Specifically, the polypeptide fragment in f) specifically refers to: a polypeptide fragment with one or more (specifically 1-50, 1-30, 1-20, 1-10, 1-5, 1-3, 1, 2 or 3) amino acid substitution, deletion or addition of the amino acid sequence shown in SEQ ID NO: 2, or a polypeptide fragment with one or more (specifically 1-50, 1-30, 1-20, 1-10, 1-5, 1-3, 1, 5 or 3) amino acids added at the N-terminus and/or C-terminus and the function of the polypeptide fragment with the amino acid sequence shown in SEQ ID NO: 5. For example, the CIITA fragment typically has dual or multifunctional domains, primarily acting as a transcriptional activator and playing a key role in the expression of major histocompatibility complex (MHC) class II genes. The amino acid sequence in f) may have more than 90%, more than 93%, more than 95%, more than 97% or more than 99% sequence identity with SEQ ID NO: 5. The CIITA fragment usually originated from human. The amino acid sequence of CIITA protein is as follows:

(SEQ ID NO: 5)
MRCLAPRPAGSYLSEPQGSSQCATMELGPLEGGYLELLNSDADPLCLY
HFYDQMDLAGEEEIELYSEPDTDTINCDQFSRLLCDMEGDEETREAYA
NIAELDQYVFQDSQLEGLSKDIFKHIGPDEVIGESMEMPAEVGQKSQK
RPFPEELPADLKHWKPAEPPTVVTGSLLVRPVSDCSTLPCLPLPALFN
QEPASGQMRLEKTDQIPMPFSSSSLSCLNLPEGPIQFVPTISTLPHGL
WQISEAGTGVSSIFIYHGEVPQASQVPPPSGFTVHGLPTSPDRPGSTS
PFAPSATDLPSMPEPALTSRANMTEHKTSPTQCPAAGEVSNKLPKWPE
PVEQFYRSLQDTYGAEPAGPDGILVEVDLVQARLERSSSKSLERELAT
PDWAERQLAQGGLAEVLLAAKEHRRPRETRVIAVLGKAGQGKSYWAGA
VSRAWACGRLPQYDFVFSVPCHCLNRPGDAYGLQDLLFSLGPQPLVAA
DEVFSHILKRPDRVLLILDGFEELEAQDGFLHSTCGPAPAEPCSLRGL
LAGLFQKKLLRGCTLLLTARPRGRLVQSLSKADALFELSGFSMEQAQA
YVMRYFESSGMTEHQDRALTLLRDRPLLLSHSHSPTLCRAVCQLSEAL
LELGEDAKLPSTLTGLYVGLLGRAALDSPPGALAELAKLAWELGRRHQ
STLQEDQFPSADVRTWAMAKGLVQHPPRAAESELAFPSFLLQCFLGAL
WLALSGEIKDKELPQYLALTPRKKRPYDNWLEGVPRFLAGLIFQPPAR
CLGALLGPSAAASVDRKQKVLARYLKRLQPGTLRARQLLELLHCAHEA
EEAGIWQHVVQELPGRLSFLGTRLTPPDAHVLGKALEAAGQDFSLDLR
STGICPSGLGSLVGLSCVTRFRAALSDTVALWESLQQHGETKLLQAAE
EKFTIEPFKAKSLKDVEDLGKLVQTQRTRSSSEDTAGELPAVRDLKKL
EFALGPVSGPQAFPKLVRILTAFSSLQHLDLDALSENKIGDEGVSQLS
ATFPQLKSLETLNLSQNNITDLGAYKLAEALPSLAASLLRLSLYNNCI
CDVGAESLARVLPDMVSLRVMDVQYNKFTAAGAQQLAASLRRCPHVET
LAMWTPTIPFSVQEHLQQQDSRISLR

In the constructing method provided by the present disclosure, the method for not expressing CIITA specifically comprises: knocking out exon 3 of the CIITA gene in the genome of human pluripotent stem cells; preferably, the knockout is performed by gene editing methods; more preferably, a gRNA with the nucleotide sequence shown in SEQ ID NO: 11 (GGGAGGCTTATGCCAATAT) is used for gene editing.

In the constructing method provided by the present disclosure, the second B2M fragment may comprise: g) a polypeptide fragment with an amino acid sequence as shown in SEQ ID NO: 2; or h) a polypeptide fragment with an amino acid sequence of more than 90% sequence identity with SEQ ID NO: 2 and the function of the polypeptide fragment in g).

The amino acid sequence of second B2M fragment is as follows:

(SEQ ID NO: 2)
MSRSVALAVLALLSLSGLEAIQRTPKIQVYSRHPAENGKSNFLNCYVS
GFHPSDIEVDLLKNGERIEKVEHSDLSFSKDWSFYLLYYTEFTPTEKD
EYACRVNHVTLSQPKIVKWDRDM

Specifically, the polypeptide fragment in h) specifically refers to: a polypeptide fragment with one or more (specifically 1-50, 1-30, 1-20, 1-10, 1-5, 1-3, 1, 2 or 3) amino acid substitution, deletion or addition of the amino acid sequence shown in SEQ ID NO: 2, or a polypeptide fragment with one or more (specifically 1-50, 1-30, 1-20, 1-10, 1-5, 1-3, 1, 2 or 3) amino acids added at the N-terminus and/or C-terminus and the function of the polypeptide fragment with the amino acid sequence shown in SEQ ID NO: 2. For example, the second B2M fragment usually has a β-pleated sheet structure and it mainly has the function of binding to the major histocompatibility complex class I heavy chain through non-covalent bonds. The amino acid sequence in h) may have more than 90%, more than 93%, more than 95%, more than 97% or more than 99% sequence identity with SEQ ID NO: 2. The second B2M fragment usually originated from human.

In the constructing method provided by the present disclosure, the HLA-G5 fragment may comprise: i) a polypeptide fragment with an amino acid sequence as shown in SEQ ID NO: 6; or j) a polypeptide fragment with an amino acid sequence of more than 90% sequence identity with SEQ ID NO: 6 and the function of the polypeptide fragment in i).

The amino acid sequence of HLA-G5 is as follows:

(SEQ ID NO: 6)
GSHSMRYFSAAVSRPGRGEPRFIAMGYVDDTQFVRFDSDSACPRMEPR
APWVEQEGPEYWEEETRNTKAHAQTDRMNLQTLRGYYNQSEASSHTLQ
WMIGCDLGSDGRLLRGYEQYAYDGKDYLALNEDLRSWTAADTAAQISK
RKCEAANVAEQRRAYLEGTCVEWLHRYLENGKEMLQRADPPKTHVTHH
PVFDYEATLRCWALGFYPAEIILTWQRDGEDQTQDVELVETRPAGDGT
FQKWAAVVVPSGEEQRYTCHVQHEGLPEPLMLRWSKEGDGGIMSVRES
RSLSEDL*

Specifically, the polypeptide fragment in j) specifically refers to: a polypeptide fragment with one or more (specifically 1-50, 1-30, 1-20, 1-10, 1-5, 1-3, 1, 2 or 3) amino acid substitution, deletion or addition of the amino acid sequence shown in SEQ ID NO: 6, or a polypeptide fragment with one or more (specifically 1-50, 1-30, 1-20, 1-10, 1-5, 1-3, 1, 6 or 3) amino acids added at the N-terminus and/or C-terminus and the function of the polypeptide fragment with the amino acid sequence shown in SEQ ID NO: 6. For example, the HLA-G5 fragment usually has a complete α-heavy chain structure. Unlike classical HLA I molecules, it mainly exhibits immunosuppressive functions (for example, binding to inhibitory receptors, thereby regulating immune responses mediated by B cells, T cells, NK cells, and APC cells, wherein the inhibitory receptors mainly comprise ILT2/CD85j/LILRB1, ILT4/CD85d/LILRB2, and KIR2DL4/CD158d, etc.). The amino acid sequence in j) may have more than 90%, more than 93%, more than 95%, more than 97% or more than 99% sequence identity with SEQ ID NO: 6. The HLA-G5 fragment usually originated from human.

In the constructing method provided by the present disclosure, the B2M-HLA-G5 fusion protein may also comprise a second flexible linker peptide, and the second flexible linker peptide is usually located between the HLA-G5 fragment and the second B2M fragment. The second flexible linker peptide is typically a flexible polypeptide of appropriate length consisting of glycine (G), serine (S) and/or alanine (A), allowing adjacent protein domains to move relative to each other freely. For example, the amino acid sequence of the second flexible linker peptide can comprise sequences such as (GS)ₙ, (GGS)ₙ, (GGSG)ₙ, (GGGS)ₙA, (GGGGS)ₙA, (GGGGA)ₙA, (GGGGG)ₙA, wherein n is an integer selected from 1 to 10. In some specific examples of the present disclosure, the length of the amino acid sequence of the second flexible linker peptide may be 5-26. In some specific examples of the present disclosure, the second flexible linker peptide may comprise a polypeptide fragment with an amino acid sequence as shown in SEQ ID NO: 5. In other specific examples of the present disclosure, the B2M-HLA-G5 fusion protein comprises, from the N-terminus to the C-terminus, the second B2M fragment and the HLA-G5 fragment, wherein the B2M-HLA-G5 fusion protein comprises a polypeptide fragment with the amino acid sequence shown in SEQ ID NO: 8.

The amino acid sequence of the flexible (G₄S)₄ linker peptide (the second flexible linker peptide) is as follows:

(SEQ ID NO: 3)
GlyGlyGlyGlySerGlyGlyGlyGlySerGlyGlyGly
GlySerGlyGlyGlyGlySer

The amino acid sequence of B2M-HLA-G5 fusion protein is as follows:

(SEQ ID NO: 8)
MSRSVALAVLALLSLSGLEAIQRTPKIQVYSRHPAENGKSNFLNCYVS
GFHPSDIEVDLLKNGERIEKVEHSDLSFSKDWSFYLLYYTEFTPTEKD
EYACRVNHVTLSQPKIVKWDRDMGGGGSGGGGSGGGGSGGGGSGSHSM
RYFSAAVSRPGRGEPRFIAMGYVDDTQFVRFDSDSACPRMEPRAPWVE
QEGPEYWEEETRNTKAHAQTDRMNLQTLRGYYNQSEASSHTLQWMIGC
DLGSDGRLLRGYEQYAYDGKDYLALNEDLRSWTAADTAAQISKRKCEA
ANVAEQRRAYLEGTCVEWLHRYLENGKEMLQRADPPKTHVTHHPVFDY
EATLRCWALGFYPAEIILTWQRDGEDQTQDVELVETRPAGDGTFQKWA
AVVVPSGEEQRYTCHVQHEGLPEPLMLRWSKEGDGGIMSVRESRSLSE
DL*

The nucleic acid sequence encoding B2M-HLA-G5 fusion protein is as follows:

(SEQ ID NO: 10)
ATGTCTCGCtccgtggccttagctgtgctcgcgctactctctctttct
ggcctggaggctatccagcgtactccaaagattcaggtttactcacgt
catccagcagagaatggaaagtcaaatttcctgaattgctatgtgtct
gggtttcatccatccgacattgaagttgacttactgaagaatggagag
agaattgaaaaagtggagcattcagacttgtctttcagcaaggactgg
tctttctatctcttgtactacactgaattcacccccactgaaaaagat
gagtatgcctgccgtgtgaaccatgtgactttgtcacagcccaagATA
GTTAAGTGGGATCGAGACATGGGTGGAGGTGGAAGTGGTGGAGGTGGA
AGTGGTGGAGGTGGAAGTGGTGGAGGTGGaagtGGCTCCCACTCCATG
AGGTATttcagcgccgccgtgtcccggcccggccgcggggagccccgc
ttcatcgccatgggctacgtggacgacacgcagttcgtgcggttcgac
agcgactcggcgtgtccgaggatggagccgcgggcgccgtgggtggag
caggaggggccggagtattgggaagaggagacacggaacaccaaggcc
cacgcacagactgacagaatgaacctgcagaccctgcgcggctactac
aaccagagcgaggccagttctcacaccctccagtggatgattggctgc
gacctggggtccgacggacgcctcctccgcgggtatgaacagtatgcc
tacgatggcaaggattacctcgccctgaacgaggacctgcgctcctgg
accgcagcggacactgcggctcagatctccaagcgcaagtgtgaggcg
gccaatgtggctgaacaaaggagagcctacctggagggcacgtgcgtg
gagtggctccacagatacctggagaacgggaaggagatgctgcagcgc
gcggacccccccaagacacacgtgacccaccaccctgtctttgactat
gaggccaccctgaggtgctgggccctgggcttctaccctgcggagatc
atactgacctggcagcgggatggggaggaccagacccaggacgtggag
ctcgtggagaccaggcctgcaggggatggaaccttccagaagtgggca
gctgtggtggtgccttctggagaggagcagagatacacgtgccatgtg
cagcatgaggggctgccggagcccctcatgctgagatggagtaaggag
ggagatggaggcatcatgtctgttagggaaagcaggagcctctctgaa
gacCTTTAA

In the constructing method provided by the present disclosure, the method of integrating exogenous nucleic acid encoding B2M-HLA-G5 fusion protein into the genome of human pluripotent stem cells can specifically comprise: integrating the nucleic acid encoding the B2M-HLA-G5 fusion protein into the genome of human pluripotent stem cells via a lentiviral vector, allowing the cells to express the B2M-HLA-G5 fusion protein.

The constructing method provided by the present disclosure successfully integrates the HLA-G1 fragment sequence at two endogenous B2M loci in human pluripotent stem cells, knocks out the CIITA fragment sequence, and integrates the sequence encoding the B2M-HLA-G5 fusion protein into the genome of human pluripotent stem cells via a lentiviral vector. The resulting human pluripotent stem cell lines do not express endogenous HLA-A, -B, or -C proteins on the cell surface. At the same time, CIITA was knocked out, resulting in pluripotent stem cells with excellent immune compatibility, unaffected pluripotency and proliferative ability. Furthermore, these pluripotent stem cells can serve as a source of cells for cell transplantation.

In some embodiments, the disruption of gene expression in pluripotent stem cells is generally referred to as “gene knockout” in this article. Usually, the methods used to achieve the two knockout are the same. Methods to achieve gene knockout comprise but are not limited to ZFN, TALEN, and CRISPR/Cas9 methods. Among them, particularly useful is the implementation of CRISPR/Cas9 to disrupt the gene. In some cases, CRISPR/Cas9 is used to introduce small deletions/insertions into the coding region of a gene, resulting in the production of non-functional proteins, such as by causing frameshift mutations that introduce premature stop codons and lead to truncated, non-functional proteins.

To prevent the expression of CIITA or B2M as target genes, other methods for downregulating target gene expression are also optional. One alternative approach involves using target gene-specific interference RNA molecules (such as siRNA, shRNA, miRNA, etc.). Based on the CIITA or B2M sequence information provided in the present disclosure, such interference RNA molecules can be prepared. The interference RNA can be delivered into cells using suitable transfection reagents or other known techniques in the field. The length of siRNAs is typically around 21 nucleotides (e.g., 21-23 nucleotides). After small RNA or RNAi is introduced into the cells, the sequence is transferred to an enzyme complex known as RISC (RNA-induced silencing complex), then the RISC recognizes the target and cleaves it with a nuclease. As another optional embodiment, shRNA technology can be used for interference. shRNA is an RNA sequence that can form a tight hairpin loop and it can silence gene expression via RNA interference. Vectors are used when using shRNA to introduce the RNA into the cells and promoters (such as U6) are used for ensuring continuous expression of shRNA. In another alternative embodiment, antisense compounds are used for specifically hybridizing with the target gene to regulate gene expression. Oligomers specifically hybridizing with their target nucleic acids interfere with the normal function of the nucleic acids. The regulation of the target nucleic acid's function by compounds that specifically hybridize with the target nucleic acid is generally referred to as “antisense.” As another alternative embodiment, homologous recombination can be used to specifically target the gene of interest, leading to defective or absent expression. Nevertheless, methods based on CRISPR are the preferred embodiment. Those skilled in the art will understand that the human pluripotent stem cells constructed using the methods provided in the present disclosure are also within the scope of the present disclosure.

The disclosure is further described below in conjunction with specific embodiments. It should be understood that these examples are merely for illustrative purposes rather than intended to limit the scope of the disclosure. The experimental methods without specifying the specific conditions in the following examples generally used the conventional conditions, such as those described in J. Sambrook, Molecular Cloning: A Laboratory Manual (3^{rd} ed. Science Press, 2002) or followed the manufacturer's recommendation.

Unless otherwise specified, the following data was statistically analyzed using GraphPad software. The comparison between two groups of data was tested using the t test method to see whether there is statistical significance; the comparison between 3-4 groups of data was performed by one-way ANOVA, followed by post hoc testing with the Dunnett method. P-values less than 0.05 were considered statistically significant.

### Example 1. Construction of B2M/CIITA Double Knockout hPSCs (DKO-hPSCs)

In the subsequent examples of the present disclosure, the hPSCs used are derived from human embryonic stem cells, specifically the H9 cell line.

To compare the immune compatibility of CIITA^{-/-}B2Mm/sHLAG-hPSCs-derived cells, B2M/CIITA double knockout hPSCs were constructed as a control. Gene editing methods (CRISPR/Cas9 system were used in this example) were employed to simultaneously knockout the B2M and CIITA genes.

Based on the transcript information of the B2M and CIITA genes, after analysis and research, the inventors optimized the sequences suitable for the gene editing locations and designed gRNAs targeting these genomic sites. The gRNA sequences are shown in Table 1. The gRNAs were separately cloned into U6 promoter-driven plasmids to obtain the gRNA expressive plasmids, gRNA-B2M and gRNA-CIITA. For constructing the DKO-hPSCs, the gRNA-B2M plasmid and Cas9-T2A-GFP (Addgene #44719) plasmid were co-transfected into hPSCs. After 4 days of culture, low-density single-cell plating was performed. After single clone growth, the clones were manually picked and subjected to genomic DNA PCR and sequencing analysis. Clones with non-triplet base deletions or insertions were selected as B2M knockout hPSCs.

On this basis, CIITA knockout hPSCs were further constructed using the same method, but the gRNA plasmid was replaced with the gRNA-CIITA plasmid. Single clones with CIITA knockout were further selected. By this method, the gene-edited hPSCs were constructed. Clones exhibiting frameshift mutations caused by non-triplet base deletions or insertions were selected as B2M/CIITA double knockout hPSCs (DKO-hPSCs) (FIG. 2A).

TABLE 1
gRNA sequences targeting B2M and CIITA sites

| Sites | gRNA sequence |
|---|---|
| B2M | AAGATTCAGGTTTACTCAC(SEQ ID NO: 7) |
| CIITA | GGGAGGCTTATGCCAATAT(SEQ ID NO: 11) |

### Example 2. Construction of CIITA^{-/-}B2Mm/sHLAG-hPSCs

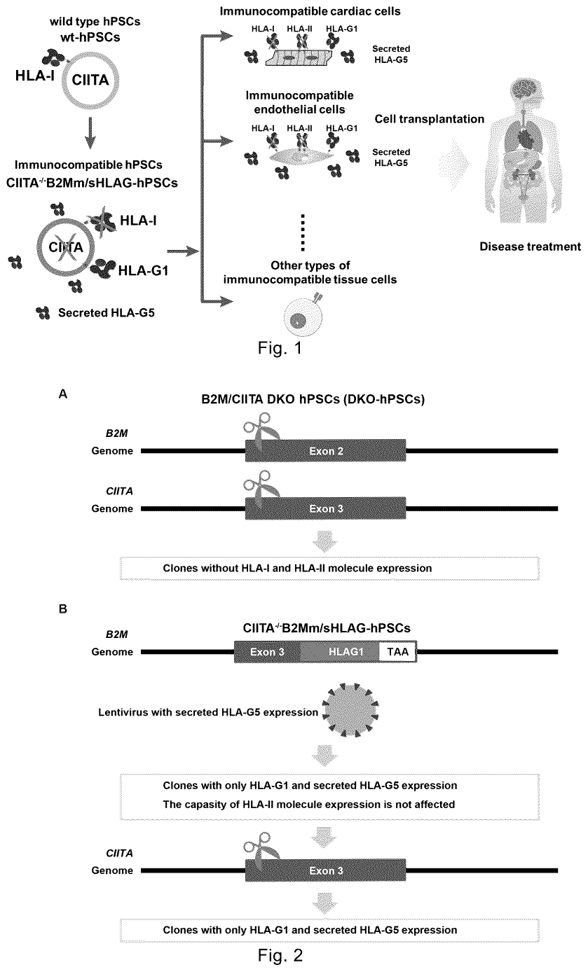

The method for constructing CIITA^{-/-}B2Mm/sHLAG-hPSCs is based on the cells constructed in patent application CN113528448A, gene editing methods (CRISPR/Cas9 were used in this example) were employed to knockout the CIITA gene, specifically (as shown in FIG. 1):

In wild-type hPSCs, the CRISPR/Cas9 gene editing method was used to insert a gene encoding HLA-G1 linked with a flexible (G₄S)₄ linker peptide (recombinant expression of B2M-HLA-G1 fusion protein, as shown in SEQ ID NO: 4) before the stop codon of the B2M gene coding frame. The resulting cell line only expresses the HLA-G1/B2M complex. Other HLA I family molecules cannot be expressed normally on the cell membrane due to the lack of B2M subunits. Next, the encoding frame of B2M-(G₄S)₄-HLA-G5 was introduced into the genome of the above cell line: the B2M CDS sequence (SEQ ID NO: 2), a sequence encoding the (Gly₄Ser)₄ flexible linker peptide, and a sequence encoding the HLA-G5 heavy chain were integrated into the BamHI and MluI sites of the puromycin-resistant lentiviral vector pLVX-CAG-Puro. The recombinant lentiviral vector was then introduced into the cells to obtain B2Mm/sHLAG hPSCs (as described in CN113528448A).

Furthermore, using gene editing methods (CRISPR/Cas9 system was used in this example), the CIITA gene was knocked out. The gRNA-CIITA plasmid (with gRNA sequence as shown in Table 1) and Cas9-T2A-GFP plasmid were transfected into the cells by LONZA nucleofection, and single clones were picked for DNA PCR sequencing. Clones with non-triplet base frameshift mutations were selected to obtain CIITA knockout B2Mm/sHLAG hPSCs, thus immunocompatible hPSCs were constructed, named CIITA^{-/-}B2Mm/sHLAG-hPSCs (FIG. 2B). In clones used in subsequent examples, a deletion of five bases after the mRNA sequence “ATGGAAGGTGATGAAGAGAC” and an insertion of one base after “ATGGAAGGTGATGAAGAGACCAGGG” led to a frameshift mutation at position 90 or 92 of the CIITA protein, causing premature termination at position 124 or 126 of the CIITA protein.

These cells can further differentiate into tissue-specific cell lines, such as cardiomyocytes, endothelial cells, neural cells, NK cells, and other immunocompatible functional cells.

### Example 3. Identification of Pluripotent Markers

To assess self-renewal capability of DKO-hPSCs and CIITA^{-/-}B2Mm/sHLAG-hPSCs, wild-type hPSCs (wt-hPSCs) were used as the control. The expression of pluripotent markers in wt-hPSCs, DKO-hPSCs and CIITA−/− B2Mm/sHLAG-hPSCs were identified by flow cytometry and immunofluorescence.

### Flow Cytometry for Identification:

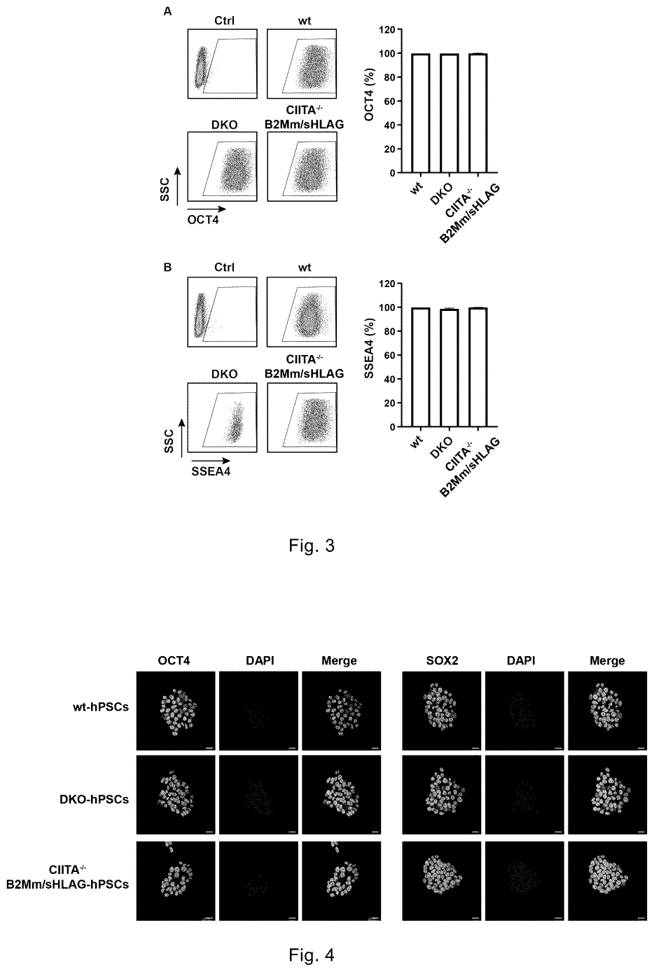

Cultured wt-hPSCs, DKO-hPSCs and CIITA−/−B2Mm/sHLAG-hPSCs were grown to 70% confluence, digested with Accutase to generate single cells, centrifuged, fixed, permeabilized, and incubated with antibodies of hPSCs pluripotent marker OCT4 and SSEA4. Secondary antibodies conjugated with APC fluorophores were used for staining and flow cytometry analysis was conducted. Isotype control antibodies were used for the control group. The results are shown in FIGS. 3A-B.

### Cell Immunofluorescence for Identification:

Cultured wt-hPSCs, DKO-hPSCs and CIITA−/−B2Mm/sHLAG-hPSCs were allowed to grow into colonies of appropriate size. After removing the culture medium, the cells were fixed, permeabilized and incubated with antibodies of pluripotent marker OCT4 and SOX2. Secondary antibodies conjugated with Alexa 488 fluorophores were used for staining and images were collected using a laser confocal microscope. The results are shown in FIG. 4.

As shown in FIG. 3, positive expression rates of pluripotency marker OCT4 and SSEA4 in all three cell types were above 98%. As shown in FIG. 4, pluripotent marker OCT4 and SOX2 exhibited good nuclear localization characteristics.

These data demonstrate that the obtained DKO-hPSCs and CIITA^{-/-}B2Mm/sHLAG-hPSCs show no difference in pluripotent marker expression compared to wt-hPSCs, indicating that their self-renewal capabilities are well.

### Example 4. Identification of HLA Molecule Expression

To identify whether the cells constructed using the above methods express HLA molecules, the inventors first performed flow cytometry analysis.

### Flow Cytometry for Identification:

wt-hPSCs, DKO-hPSCs and CIITA−/−B2Mm/sHLAG-hPSCs cells were cultured in the presence of interferon gamma (IFN-γ) at a final concentration of 100 ng/ml. After 48 hours of culture, the cells were digested into single cells using Accutase, centrifuged and collected for detection of HLA molecule expression. Since there are numerous members in the HLA I and HLA II families, HLA-A, -B, -C (representative members of the HLA I family) and HLA-DR (a representative member of the HLA II family) were used to characterize the expression of HLA I and HLA II molecules in wt-hPSCs, DKO-hPSCs and CIITA−/−B2Mm/sHLAG-hPSCs. HLA-A/B/C antibodies were conjugated to FITC fluorophores and HLA-DR antibodies were conjugated to PE fluorophores. Single cells were incubated with the corresponding antibodies and analyzed by flow cytometry to detect HLA I and HLA II molecule expression. HLA-G1 expression was also identified using the same flow cytometry method.

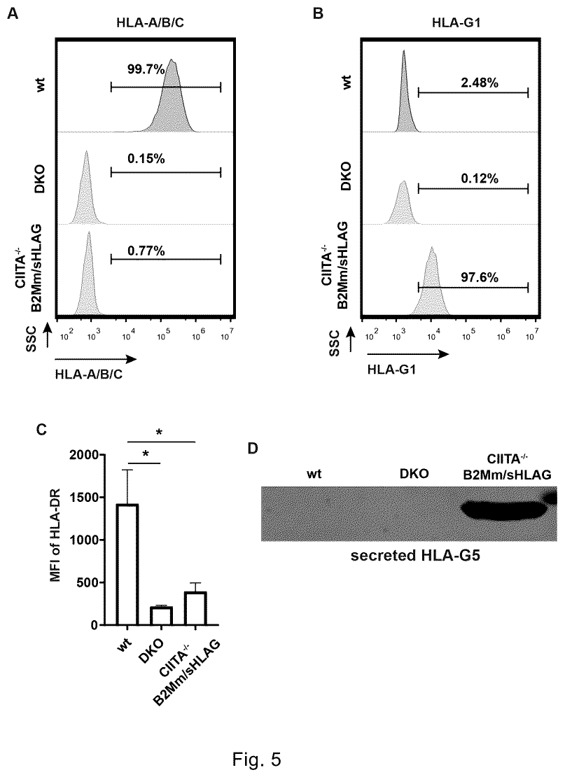

HLA-A, -B, -C are classic members of the HLA I family. When comparing the expression of HLA-A, -B, -C in wt-hPSCs, DKO-hPSCs and CIITA^{-/-}B2Mm/sHLAG-hPSCs, it was found that HLA-A, -B, -C were only expressed in wt-hPSCs, and no expression was detected in DKO-hPSCs or CIITA−/−B2Mm/sHLAG-hPSCs (FIG. 5A). In contrast, the immunosuppressive HLA-G1 molecule was only expressed in CIITA^{-/-}B2Mm/sHLAG-hPSCs (FIG. 5B). After treating the hPSCs with interferon gamma (IFN-γ) for 48 hours, flow cytometry analysis of HLA-DR expression showed that the fluorescence signal in wt-hPSCs was significantly higher than that in DKO-hPSCs and CIITA^{-/-}B2Mm/sHLAG-hPSCs (FIG. 5C).

The expression of secreted HLA-G5 was identified by Western Blot.

### Secreted HLA-G5 Expression Identified by Western Blot:

wt-hPSCs, DKO-hPSCs and CIITA^{-/-}B2Mm/sHLAG-hPSCs were cultured in hPSC medium until 50% confluence. The medium was then replaced with DMEM/F12 basic medium for 24 hours. The culture supernatant was collected, filtered through a 0.25 μm filter to remove cell debris, and concentrated using a 10 kDa ultrafiltration tube. Before analyzing by Western blot, loading buffer (comprising 0-mercaptoethanol) was added to a final concentration of 1× and the proteins were denatured in a 95 °C water bath for 10 minutes. The proteins were separated by 10% SDS-PAGE gel electrophoresis and the antibody used for detection was 5A6G7.

The results showed that CIITA^{-/-}B2Mm/sHLAG-hPSCs could express HLA-G5, while wt-hPSCs and DKO-hPSCs did not express HLA-G5 (FIG. 5D).

### Example 5. Induced Differentiation and Identification of Endothelial and Cardiomyocytes

hPSCs can be directed to differentiate into various tissue-type cells by in vitro induction. In this example, hPSCs were differentiated into endothelial cells and cardiomyocytes by the inventors.

### 1. Differentiation of Endothelial Cells

wt-hPSCs, DKO-hPSCs and CIITA^{-/-}B2Mm/sHLAG-hPSCs were cultured until 100% confluence and then switched to differentiation medium with RPMI1640+0.2% BSA and 6 μM CHIR99021 for 2 days. The medium was then changed to RPMI1640+0.2% BSA+50 ng/ml bFGF for 1 day, followed by RPMI1640+0.2% BSA+50 ng/ml VEGF+50 ng/ml BMP4 for 2 days. On the fifth day, endothelial cells were purified using Miltenyi CD144 (VE-Cadherin) MicroBeads. Purified cells were further cultured in ECM medium.

The differentiated cells (endothelial cells on the 6th day; cardiomyocytes on the 14th day) were digested with Accutase into single cells and cell clumps were removed using a 70 μm filter. Endothelial cells were detected by incubating directly with APC-conjugated CD144 antibody. Cardiomyocytes were fixed and permeabilized, then incubated with primary antibody against cardiac troponin T (cTNT) and secondary antibody conjugated to APC against mouse IgG for detection.

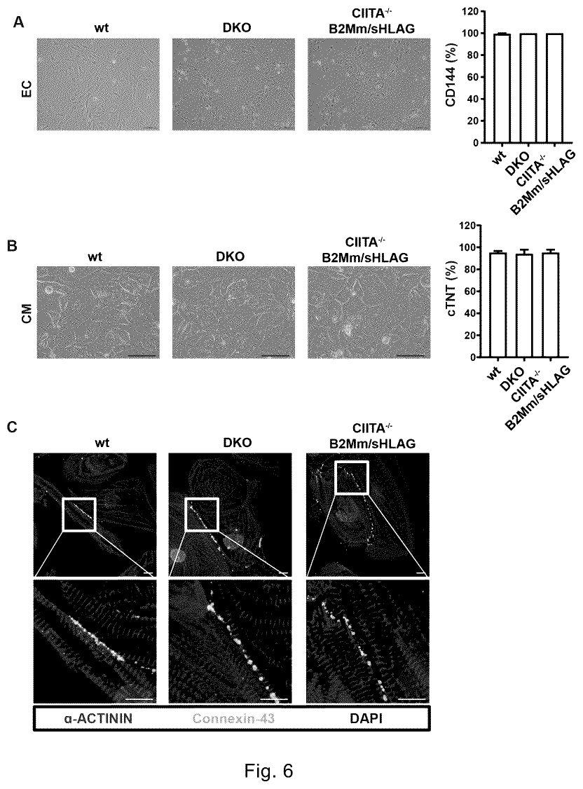

The results showed that in the endothelial cell differentiation, purified endothelial cells exhibited good endothelial morphology and a very high purity of CD144 expression (FIG. 6A). Thus, immunocompatible endothelial cells were successfully obtained.

### 2. Differentiation of Cardiomyocytes

wt-hPSCs, DKO-hPSCs and CIITA^{-/-}B2Mm/sHLAG-hPSCs were cultured until 100% confluence, and then switched to differentiation medium with RPMI1640+0.2% BSA+6 μM CHIR99021 for 2 days. The medium was then changed to RPMI1640+0.2% BSA+5 μM IWR-1 for 2 days, and subsequently, replaced with RPMI1640+0.2% BSA every 2 days. On the 7th day, beating cardiomyocytes began to appear. These were named as wt-CMs, DKO-CMs and CIITA−/−B2Mm/sHLAG-hPSCs, depending on the originating hPSCs.

Cell immunofluorescence for cardiomyocyte identification was the same as described for hPSCs pluripotent markers in Example 3. The primary antibody was replaced with antibodies of cardiac troponin T (cTNT) and gap junction Connexin 43.

In differentiated cardiomyocytes, sarcomeric protein alpha-ACTININ staining showed that cardiomyocytes derived from all three hPSCs had well-organized sarcomeric structures, and good gap junctions (Connexin-43) were formed between the cells (FIG. 6B). Expression of sarcomeric protein α-ACTININ and gap protein Connexin-43 in differentiated cardiomyocytes was measured by immunofluorescence, as shown in FIG. 6C.

Thus, immunocompatible cardiomyocytes (CIITA B2Mm/sHLAG-CMs) were successfully obtained.

### Example 6. Immune Recognition and Killing Effects

To investigate whether CIITA^{-/-}B2Mm/sHLAG-hPSCs and their derived cells have the ability to escape recognition and killing by immune cells, the inventors used an in vitro co-culture system with immune cells (PBMCs or NK92) and cardiomyocytes derived from CIITA^{-/-}B2Mm/sHLAG-hPSCs. T cells were tested for activation markers, proliferation, killing effect on target cells and secreted IFN-γ. NK cells were tested for target cell recognition, killing effects and secreted IFN-γ.

### 1. Detection of Activation Markers on T Cells

T cells are activated by mismatched HLA molecules. Therefore, wt-CMs as a control and CIITA^{-/-}B2Mm/sHLAG-hPSCs were used to compare the activation of T cells. wt-CMs and CIITA−/− B2Mm/sHLAG-hPSCs were plated on a 96-well plate. After 24 hours, IFN-γ was added at a final concentration of 100 ng/ml and incubated for 48 hours to stimulate HLA molecule expression. After removing IFN-γ, primary human peripheral blood mononuclear cells (PBMCs) (with a 1:3 ratio of PBMCs to cardiomyocytes) were added for 48-hour co-culture. The PBMCs were then collected, incubated with antibodies of T cell marker CD3 and early T cell activation marker CD69, followed by flow cytometry analysis. The activation of T cells was evaluated by comparing the percentage of CD69-positive cells among CD3-positive cells between wt-CMs and CIITA^{-/-}B2Mm/sHLAG-hPSCs.

### 2. Detection of T Cell Proliferation

The method for preliminary treatment was the same as the “Detection of activation markers on T cells”. Before co-culturing, PBMCs were labeled with CFSE fluorescent dye and then co-cultured with cardiomyocytes for 7 days. Afterward, PBMCs were collected and incubated with antibody of T cell marker CD3, followed by flow cytometry analysis. CFSE does not leak from the labeled cells but is diluted during cell division. Therefore, T cells that have proliferated will show reduced CFSE fluorescence intensity. The comparison of the percentage of CD3-positive cells with low CFSE signal between wt-CMs and CIITA^{-/-}B2Mm/sHLAG-hPSCs was used to assess the proliferation of T cells.

### 3. Detection of Killing Effects on Target Cells by T Cells

The method for preliminary treatment was the same as the “Detection of activation markers on T cells”. PBMCs were co-cultured with wt-CMs or CIITA^{-/-}B2Mm/sHLAG-hPSCs for 3-4 days. Afterwards, the co-culture supernatant was collected, centrifuged at 300 g for 3 minutes to remove cells and cell debris. Lactate dehydrogenase (LDH) activity in the supernatant was measured using the Biyuntian LDH assay kit to assess cell damage.

### 4. Detection of IFN-γ Secreted by PBMC

The method for preliminary treatment was the same as the “Detection of activation markers on T cells”. PBMCs were co-cultured with wt-CMs or CIITA^{-/-}B2Mm/sHLAG-hPSCs for 3-4 days. Afterwards, the co-culture supernatant was collected, centrifuged at 300 g for 3 minutes to remove cells and cell debris. The IFN-γ level in the supernatant was measured using a IFN-γ ELISA kit.

### 5. Detection of Killing Effects on Target Cells by NK Cells

NK cells can recognize cells that lack HLA molecule expression. In this example, DKO-CMs were used as a control. DKO-CMs and CIITA^{-/-}B2Mm/sHLAG-hPSCs were plated on a 96-well plate, and after 24 hours, NK-92 cells were added at a ratio of CMs:NK-92=10:1, 3:1 or 1:1, and cultured for 48 hours. The co-culture supernatant was then collected, centrifuged at 300 g for 3 minutes to remove cells and cell debris. LDH activity in the supernatant was measured using the Biyuntian LDH assay kit to assess the degree of cell damage.

### 6. Detection of IFN-γ Secreted by NK Cells

The method for preliminary treatment was the same as the “Detection of activation markers on T cells”. After 3-4 days of co-culture, the co-culture supernatant was collected, centrifuged at 300 g for 3 minutes to remove cells and cell debris. The IFN-γ level in the supernatant was measured using an IFN-γ ELISA kit.

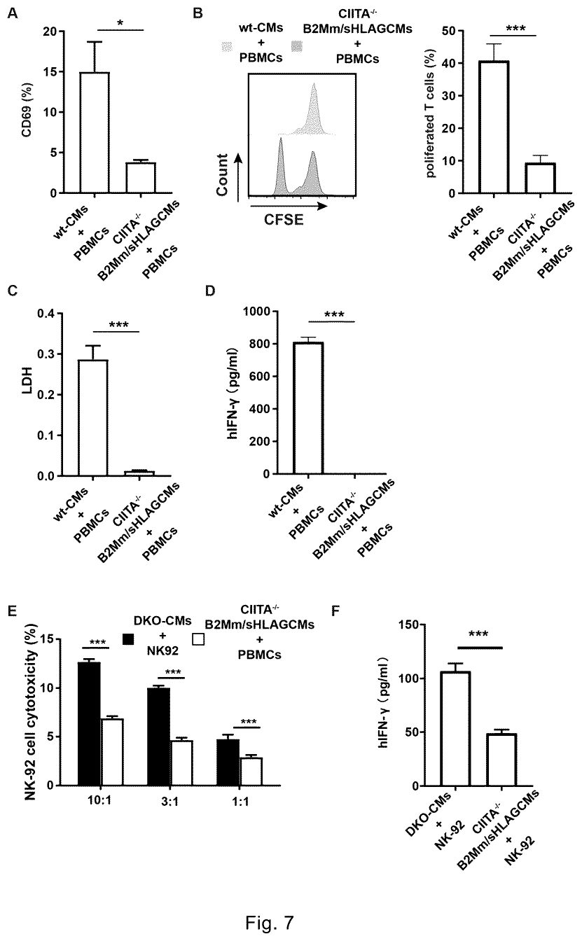

Human PBMCs were co-cultured with cardiomyocytes and the expression of early T cell activation marker CD69 was measured. The results showed that compared to wt cardiomyocytes (wt-CMs), immunocompatible cardiomyocytes (CIITA^{-/-}B2Mm/sHLAG-CMs) significantly reduced T cell activation. Besides, CIITA^{-/-}B2Mm/sHLAG-CMs had a significantly lower effect on T cell proliferation compared to wt-CMs. Simultaneously, PBMCs had a significantly lower killing effect on CIITA^{-/-}B2Mm/sHLAG-CMs than on wt-CMs and the ability of CIITA^{-/-}B2Mm/sHLAG-CMs to stimulate PBMCs to secrete IFN-γ was also significantly lower than that of wt-CMs, as shown in FIGS. 7A-B.

To test the activation effect of CIITA B2Mm/sHLAG-CMs on NK cells, DKO-CMs were used as a control. Co-culture of differentiated cardiomyocytes with NK-92 cells revealed that NK-92 cells had a significantly lower killing effect on CIITA B2Mm/sHLAG-CMs compared to DKO-CMs, and CIITA^{-/-}B2Mm/sHLAG-CMs stimulated NK-92 cells to secrete significantly lower levels of IFN-γ compared to DKO-CMs, as shown in FIGS. 7C-F.

These data demonstrate that cardiomyocytes derived from CIITA^{-/-}B2Mm/sHLAG-hPSCs have the ability to escape recognition and killing effects by T cells and NK cells.

### Example 7. Identification of Cell Residence after Myocardial Infarction Transplantation of hPSCs-Derived Cardiomyocytes

To further investigate whether CIITA^{-/-}B2Mm/sHLAG-hPSCs exhibit better retention effects in an in vivo injury environment, the inventors used humanized immune mice (Hu-mice) to construct a heart injury model (in this example, an ischemia/reperfusion injury model) and transplanted cardiomyocytes derived from different hPSCs after the modeling. The specific steps are as follows: At the 10th week of immune reconstitution, the Hu-mice heart with a significant proportion of human immune cells in the peripheral blood of the mice was subjected to ligation of the left anterior descending coronary artery and ischemia for 60 minutes, followed by reperfusion to construct myocardial ischemia/reperfusion (I/R) model. Model mice were randomly divided into four groups: I/R group, I/R+wt-CMs group, I/R+DKO-CMs group and I/R+CIITA^{-/-}B2Mm/sHLAG-CMs group. During reperfusion, cell transplantation was performed, with each mouse receiving 5×10⁵ cells of wt-CMs, DKO-CMs, or CIITA^{-/-}B2Mm/sHLAG-CMs by myocardial injection at the infarct border area.

On the 28th day after transplantation, heart samples were collected from the mice and cell residence was assessed using immunofluorescence and qRT-PCR. The method for detecting cell residence is as follows:

After 28 days of cell transplantation, hearts were collected from the mice and embedded in OCT. The hearts of mice were sliced into 12 layers from the apex to the ligation site, with a defined interval between layers, and each layer was processed for frozen sectioning. Then, immunofluorescence was performed to identify the transplanted cells and Masson staining was used to calculate the percentage of the engrafted cell area in the scar region.

Immunofluorescence: After frozen sectioning, the tissue was fixed, permeabilized and blocked, and then co-incubated with antibodies against sarcomeric protein α-ACTININ and human KU80 (C48E7, CST). Images were captured using a fluorescence microscope and statistical analysis was performed.

qRT-PCR: Heart samples from Hu-mice were collected 28 days after cell transplantation, and RNA was extracted and reverse-transcribed into cDNA. Fluorescent quantitative PCR was used to identify cell residence with human cTNT primers.

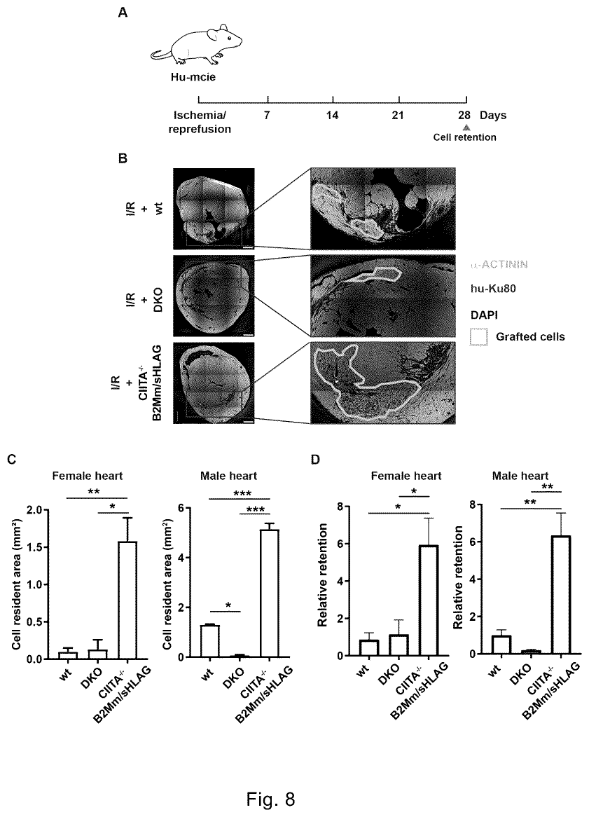

By comparing the area of cell residence, it was found that in both female and male mice, the residence area of CIITA^{-/-}B2Mm/sHLAG-CMs was significantly higher than that of wt-CMs and DKO-CMs. The results from qRT-PCR were consistent with the immunofluorescence staining results (FIGS. 8A-D).

These data demonstrate that CIITA^{-/-}B2Mm/sHLAG-CMs exhibit significantly better capabilities of long-term residence in the injury model compared to wt-CMs and DKO-CMs.

### Example 8. Repair Effects of Transplanted Cells on the Myocardial Infarction Area

Furthermore, the inventors used Masson staining to explore repair effects of transplanted cells on the injured cardiac area in mice.

Masson staining: After frozen sectioning, the tissues were fixed and stained using the Biyuntian Masson Staining Kit. The areas of re-myocardialization and scar tissue were quantified to calculate the re-myocardialization ratio.

### Re-Myocardialization Ratio=Re-Myocardial Area/Scar Area

The results showed that after myocardial injury in hearts of mice, damaged cells gradually died, with scar tissues formed. Comparison of heart sections from mice transplanted with the three types of cells revealed that after transplantation of CIITA^{-/-}B2Mm/sHLAG-CMs, myocardial tissues were formed at the injury site, compensating for the lost myocardial cells.

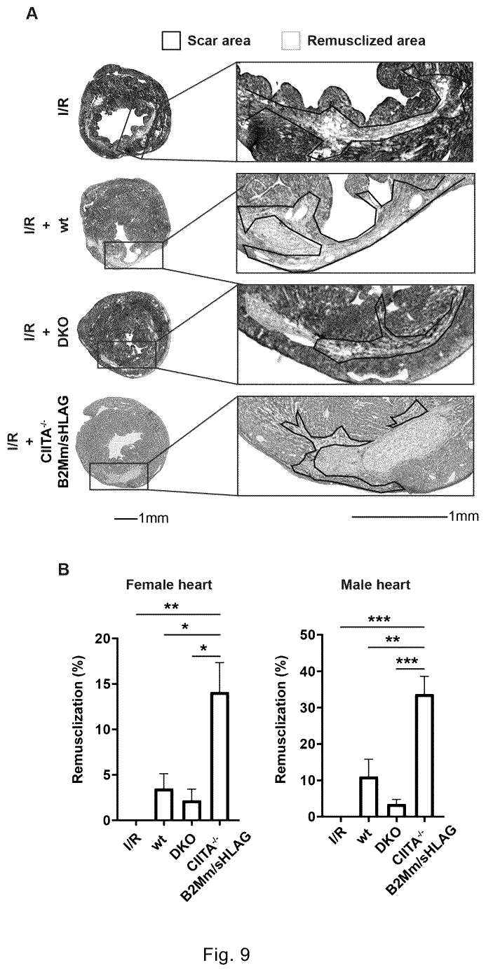

Statistical results revealed that after transplantation of CIITA^{-/-}B2Mm/sHLAG-CMs, the degree of re-myocardialization in the injury area was significantly higher compared to wt-CMs and DKO-CMs (FIGS. 9A-B).

### Example 9. Establishment and Expression of Immunocompatible Human Induced Pluripotent Stem Cells Using Non-Viral Constructs

### 1. Establishment of Immunocompatible Human Induced Pluripotent Stem Cells Using Non-Viral Constructs

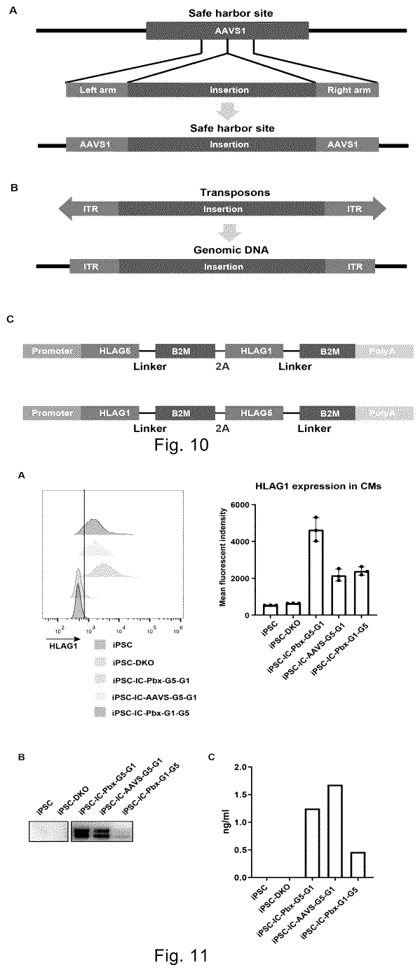

First, human iPSCs with HLA-I and HLA-II molecules deleted (i.e., hPSCs (DKO-hPSCs)) were constructed using the methods previously described. Then, gene-specific knock-in (FIG. 10A) or transposon systems (FIG. 10B) were used to introduce the relevant plasmids into the target human iPSCs. After further selection of single clones, iPSC single-cell lines expressing the target genes were obtained.

The structure of the exogenous insertion fragment is shown in FIG. 10C, wherein the N-terminal and C-terminal portions of the gene expression frame are linked by a 2A sequence. This allows the N-terminal and C-terminal proteins to separate during translation, resulting in a single expression frame that can express two independent proteins. Because of variable protein expression levels before and after the 2A structure, two forms of the expression frame were designed for screening clones with the best expression efficiency: HLAG1-B2M-2A-HLAG5-B2M and HLAG5-B2M-2A-HLAG1-B2M.

2. Expression of HLAG1 and HLAG5 in the Constructed Immunocompatible Human Induced Pluripotent Stem Cell-Derived Cardiomyocytes (iPSC-IC-CMs)

First, flow cytometry was used to detect expression levels of HLAG1 on cell membrane of cardiomyocytes derived from wild-type human iPSCs (iPSC), human iPSCs with HLA-I and HLA-II gene deletions (iPSC-DKO), gene-specific knock-in human iPSCs (iPSC-IC-AAVS) and transposon-mediated gene insertion human iPSCs (iPSC-IC-Pbx).

As shown in FIG. 11A (left), compared to iPSC-derived cardiomyocytes and iPSC-DKO-derived cardiomyocytes, iPSC-IC-AAVS-derived and iPSC-IC-Pbx-derived cardiomyocytes expressed significantly higher levels of HLAG1. The cardiomyocytes derived from iPSC single clones constructed using the PiggyBac transposon system with the HLAG5-B2M-2A-HLAG1-B2M expression frame showed the highest HLAG1 expression (FIG. 11A (right)).

To measure the secretion of HLAG5, ELISA and Western blotting were used to analyze the culture medium of cardiomyocytes. It was found that iPSC-derived cardiomyocytes with the HLAG5-B2M-2A-HLAG1-B2M expression frame exhibited higher secretion of HLAG5, while the cardiomyocytes with the HLAG1-B2M-2A-HLAG5-B2M expression frame did not show detectable HLAG5 expression in the culture supernatant (FIG. 11B).

These data suggest that different methods of gene integration and expression frame structures have a significant impact on the expression of the target gene. By combining these two methods, highly expressive clones of the exogenous gene can be obtained.

Among them, sequences of inserted fragments are as follows:

The DNA sequence of HLAG1-B2M-2A-HLAG5-B2M (SEQ ID NO: 12) is as follows:

ATGTCTCGCtccgtggccttagctgtgctcgcgctactctctctttct
ggcctggaggctatccagcgtactccaaagattcaggtttactcacgt
catccagcagagaatggaaagtcaaatttcctgaattgctatgtgtct
gggtttcatccatccgacattgaagttgacttactgaagaatggagag
agaattgaaaaagtggagcattcagacttgtctttcagcaaggactgg
tctttctatctcttgtactacactgaattcacccccactgaaaaagat
gagtatgcctgccgtgtgaaccatgtgactttgtcacagcccaagATA
GTTAAGTGGGATCGAGACATGGGTGGAGGTGGAAGTGGTGGAGGTGGA
AGTGGTGGAGGTGGAAGTGGTGGAGGTGGaagtggctcccactccatg
aggtatttcagcgccgccgtgtcccggcccggccgcggggagccccgc
ttcatcgccatgggctacgtggacgacacgcagttcgtgcggttcgac
agcgactcggcgtgtccgaggatggagccgcgggcgccgtgggtggag
caggaggggccggagtattgggaagaggagacacggaacaccaaggcc
cacgcacagactgacagaatgaacctgcagaccctgcgcggctactac
aaccagagcgaggccagttctcacaccctccagtggatgattggctgc
gacctggggtccgacggacgcctcctccgcgggtatgaacagtatgcc
tacgatggcaaggattacctcgccctgaacgaggacctgcgctcctgg
accgcagcggacactgcggctcagatctccaagcgcaagtgtgaggcg
gccaatgtggctgaacaaaggagagcctacctggagggcacgtgcgtg
gagtggctccacagatacctggagaacgggaaggagatgctgcagcgc
gcggacccccccaagacacacgtgacccaccaccctgtctttgactat
gaggccaccctgaggtgctgggccctgggcttctaccctgcggagatc
atactgacctggcagcgggatggggaggaccagacccaggacgtggag
ctcgtggagaccaggcctgcaggggatggaaccttccagaagtgggca
gctgtggtggtgccttctggagaggagcagagatacacgtgccatgtg
cagcatgaggggctgccggagcccctcatgctgagatggaagcagtct
tccctgcccaccatccccatcatgggtatcgttgctggcctggttgtc
cttgcagctgtagtcactggagctgcggtcgctgctgtgctgtggaga
aagaagagctcagatGGAAGCGGAGCTACTAACTTCAGCCTGCTGAAG
CAGGCTGGAGACGTGGAGGAGAACCCTGGACCTATGTCTCGCtccgtg
gccttagctgtgctcgcgctactctctctttctggcctggaggctatc
cagcgtactccaaagattcaggtttactcacgtcatccagcagagaat
ggaaagtcaaatttcctgaattgctatgtgtctgggtttcatccatcc
gacattgaagttgacttactgaagaatggagagagaattgaaaaagtg
gagcattcagacttgtctttcagcaaggactggtctttctatctcttg
tactacactgaattcacccccactgaaaaagatgagtatgcctgccgt
gtgaaccatgtgactttgtcacagcccaagATAGTTAAGTGGGATCGA
GACATGGGTGGAGGTGGAAGTGGTGGAGGTGGAAGTGGTGGAGGTGGA
AGTGGTGGAGGTGGaagtGGCTCCCACTCCATGAGGTATttcagcgcc
gccgtgtcccggcccggccgcggggagccccgcttcatcgccatgggc
tacgtggacgacacgcagttcgtgcggttcgacagcgactcggcgtgt
ccgaggatggagccgcgggcgccgtgggtggagcaggaggggccggag
tattgggaagaggagacacggaacaccaaggcccacgcacagactgac
agaatgaacctgcagaccctgcgcggctactacaaccagagcgaggcc
agttctcacaccctccagtggatgattggctgcgacctggggtccgac
ggacgcctcctccgcgggtatgaacagtatgcctacgatggcaaggat
tacctcgccctgaacgaggacctgcgctcctggaccgcagcggacact
gcggctcagatctccaagcgcaagtgtgaggcggccaatgtggctgaa
caaaggagagcctacctggagggcacgtgcgtggagtggctccacaga
tacctggagaacgggaaggagatgctgcagcgcgcggacccccccaag
acacacgtgacccaccaccctgtctttgactatgaggccaccctgagg
tgctgggccctgggcttctaccctgcggagatcatactgacctggcag
cgggatggggaggaccagacccaggacgtggagctcgtggagaccagg
cctgcaggggatggaaccttccagaagtgggcagctgtggtggtgcct
tctggagaggagcagagatacacgtgccatgtgcagcatgaggggctg
ccggagcccctcatgctgagatggagtaaggagggagatggaggcatc
atgtctgttagggaaagcaggagcctctctgaagacCTTTAA

The sequence of HLAG1-B2M-2A-HLAG5-B2M protein (SEQ ID NO: 11) is as follows:

MSRSVALAVLALLSLSGLEAIQRTPKIQVYSRHPAENGKSNFLNCYVS
GFHPSDIEVDLLKNGERIEKVEHSDLSFSKDWSFYLLYYTEFTPTEKD
EYACRVNHVTLSQPKIVKWDRDMGGGGSGGGGSGGGGSGGGGSGSHSM
RYFSAAVSRPGRGEPRFIAMGYVDDTQFVRFDSDSACPRMEPRAPWVE
QEGPEYWEEETRNTKAHAQTDRMNLQTLRGYYNQSEASSHTLQWMIGC
DLGSDGRLLRGYEQYAYDGKDYLALNEDLRSWTAADTAAQISKRKCEA
ANVAEQRRAYLEGTCVEWLHRYLENGKEMLQRADPPKTHVTHHPVFDY
EATLRCWALGFYPAEIILTWQRDGEDQTQDVELVETRPAGDGTFQKWA
AVVVPSGEEQRYTCHVQHEGLPEPLMLRWKQSSLPTIPIMGIVAGLVV
LAAVVTGAAVAAVLWRKKSSDGSGATNFSLLKQAGDVEENPGPMSRSV
ALAVLALLSLSGLEAIQRTPKIQVYSRHPAENGKSNFLNCYVSGFHPS
DIEVDLLKNGERIEKVEHSDLSFSKDWSFYLLYYTEFTPTEKDEYACR
VNHVTLSQPKIVKWDRDMGGGGSGGGGSGGGGSGGGGSGSHSMRYFSA
AVSRPGRGEPRFIAMGYVDDTQFVRFDSDSACPRMEPRAPWVEQEGPE
YWEEETRNTKAHAQTDRMNLQTLRGYYNQSEASSHTLQWMIGCDLGSD
GRLLRGYEQYAYDGKDYLALNEDLRSWTAADTAAQISKRKCEAANVAE
QRRAYLEGTCVEWLHRYLENGKEMLQRADPPKTHVTHHPVFDYEATLR
CWALGFYPAEIILTWQRDGEDQTQDVELVETRPAGDGTFQKWAAVVVP
SGEEQRYTCHVQHEGLPEPLMLRWSKEGDGGIMSVRESRSLSEDL*

The DNA sequence of HLAG5-B2M-2A-HLAG1-B2M (SEQ ID NO: 14) is as follows:

ATGTCTCGCtccgtggccttagctgtgctcgcgctactctctctttct
ggcctggaggctatccagcgtactccaaagattcaggtttactcacgt
catccagcagagaatggaaagtcaaatttcctgaattgctatgtgtct
gggtttcatccatccgacattgaagttgacttactgaagaatggagag
agaattgaaaaagtggagcattcagacttgtctttcagcaaggactgg
tctttctatctcttgtactacactgaattcacccccactgaaaaagat
gagtatgcctgccgtgtgaaccatgtgactttgtcacagcccaagATA
GTTAAGTGGGATCGAGACATGGGTGGAGGTGGAAGTGGTGGAGGTGGA
AGTGGTGGAGGTGGAAGTGGTGGAGGTGGaagtGGCTCCCACTCCATG
AGGTATttcagcgccgccgtgtcccggcccggccgcggggagccccgc
ttcatcgccatgggctacgtggacgacacgcagttcgtgcggttcgac
agcgactcggcgtgtccgaggatggagccgcgggcgccgtgggtggag
caggaggggccggagtattgggaagaggagacacggaacaccaaggcc
cacgcacagactgacagaatgaacctgcagaccctgcgcggctactac
aaccagagcgaggccagttctcacaccctccagtggatgattggctgc
gacctggggtccgacggacgcctcctccgcgggtatgaacagtatgcc
tacgatggcaaggattacctcgccctgaacgaggacctgcgctcctgg
accgcagcggacactgcggctcagatctccaagcgcaagtgtgaggcg
gccaatgtggctgaacaaaggagagcctacctggagggcacgtgcgtg
gagtggctccacagatacctggagaacgggaaggagatgctgcagcgc
gcggacccccccaagacacacgtgacccaccaccctgtctttgactat
gaggccaccctgaggtgctgggccctgggcttctaccctgcggagatc
atactgacctggcagcgggatggggaggaccagacccaggacgtggag
ctcgtggagaccaggcctgcaggggatggaaccttccagaagtgggca
gctgtggtggtgccttctggagaggagcagagatacacgtgccatgtg
cagcatgaggggctgccggagcccctcatgctgagatggagtaaggag
ggagatggaggcatcatgtctgttagggaaagcaggagcctctctgaa
gacCTTGGAAGCGGAGCTACTAACTTCAGCCTGCTGAAGCAGGCTGGA
GACGTGGAGGAGAACCCTGGACCTATGTCTCGCtccgtggccttagct
gtgctcgcgctactctctctttctggcctggaggctatccagcgtact
ccaaagattcaggtttactcacgtcatccagcagagaatggaaagtca
aatttcctgaattgctatgtgtctgggtttcatccatccgacattgaa
gttgacttactgaagaatggagagagaattgaaaaagtggagcattca
gacttgtctttcagcaaggactggtctttctatctcttgtactacact
gaattcacccccactgaaaaagatgagtatgcctgccgtgtgaaccat
gtgactttgtcacagcccaagATAGTTAAGTGGGATCGAGACATGGGT
GGAGGTGGAAGTGGTGGAGGTGGAAGTGGTGGAGGTGGAAGTGGTGGA
GGTGGaagtggctcccactccatgaggtatttcagcgccgccgtgtcc
cggcccggccgggggagccccgcttcatcgccatgggctacgtggacg
acacgcagttcgtgcggttcgacagcgactcggcgtgtccgaggatgg
agccgcgggcgccgtgggtggagcaggaggggccggagtattgggaag
aggagacacggaacaccaaggcccacgcacagactgacagaatgaacc
tgcagaccctgcgcggctactacaaccagagcgaggccagttctcaca
ccctccagtggatgattggctgcgacctggggtccgacggacgcctcc
tccgcgggtatgaacagtatgcctacgatggcaaggattacctcgccc
tgaacgaggacctgcgctcctggaccgcagcggacactgcggctcaga
tctccaagcgcaagtgtgaggcggccaatgtggctgaacaaaggagag
cctacctggagggcacgtgcgtggagtggctccacagatacctggaga
acgggaaggagatgctgcagcgcgcggacccccccaagacacacgtga
cccaccaccctgtctttgactatgaggccaccctgaggtgctgggccc
tgggcttctaccctgcggagatcatactgacctggcagcgggatgggg
aggaccagacccaggacgtggagctcgtggagaccaggcctgcagggg
atggaaccttccagaagtgggcagctgtggtggtgccttctggagagg
agcagagatacacgtgccatgtgcagcatgaggggctgccggagcccc
tcatgctgagatggaagcagtcttccctgcccaccatccccatcatgg
gtatcgttgctggcctggttgtccttgcagctgtagtcactggagctg
cggtcgctgctgtgctgtggagaaagaagagctcagattga

The sequence of HLAG5-B2M-2A-HLAG1-B2M Protein (SEQ ID NO: 15) is as follows:

MSRSVALAVLALLSLSGLEAIQRTPKIQVYSRHPAENGKSNFLNCYVS
GFHPSDIEVDLLKNGERIEKVEHSDLSFSKDWSFYLLYYTEFTPTEKD
EYACRVNHVTLSQPKIVKWDRDMGGGGSGGGGSGGGGSGGGGSGSHSM
RYFSAAVSRPGRGEPRFIAMGYVDDTQFVRFDSDSACPRMEPRAPWVE
QEGPEYWEEETRNTKAHAQTDRMNLQTLRGYYNQSEASSHTLQWMIGC
DLGSDGRLLRGYEQYAYDGKDYLALNEDLRSWTAADTAAQISKRKCEA
ANVAEQRRAYLEGTCVEWLHRYLENGKEMLQRADPPKTHVTHHPVFDY
EATLRCWALGFYPAEIILTWQRDGEDQTQDVELVETRPAGDGTFQKWA
AVVVPSGEEQRYTCHVQHEGLPEPLMLRWSKEGDGGIMSVRESRSLSE
DLGSGATNFSLLKQAGDVEENPGPMSRSVALAVLALLSLSGLEAIQRT
PKIQVYSRHPAENGKSNFLNCYVSGFHPSDIEVDLLKNGERIEKVEHS
DLSFSKDWSFYLLYYTEFTPTEKDEYACRVNHVTLSQPKIVKWDRDMG
GGGSGGGGSGGGGSGGGGSGSHSMRYFSAAVSRPGRGEPRFIAMGYVD
DTQFVRFDSDSACPRMEPRAPWVEQEGPEYWEEETRNTKAHAQTDRMN
LQTLRGYYNQSEASSHTLQWMIGCDLGSDGRLLRGYEQYAYDGKDYLA
LNEDLRSWTAADTAAQISKRKCEAANVAEQRRAYLEGTCVEWLHRYLE
NGKEMLQRADPPKTHVTHHPVFDYEATLRCWALGFYPAEIILTWQRDG
EDQTQDVELVETRPAGDGTFQKWAAVVVPSGEEQRYTCHVQHEGLPEP
LMLRWKQSSLPTIPIMGIVAGLVVLAAVVTGAAVAAVLWRKKSSD*

Above-described examples only show several embodiments of the present disclosure, which are described specifically and in detail. However, it should not be understood as a limiting patent scope of the present disclosure. It should be noted that those skilled in the art can make some adjustments and improvements without departing from the concept of the present disclosure and all these forms are within the scope of protection of the present disclosure. Therefore, the scope of patent protection in the present disclosure should be determined by the appended claims. Simultaneously, each reference provided herein is incorporated by reference to the same extent as if each reference was individually incorporated by reference.

1. A method for preparing immunocompatible human pluripotent stem cells, comprising modifying human pluripotent stem cells to:
(a) not express free B2M protein, express HLA-G1 and secreted HLA-G5; and
(b) not express CIITA protein.

2. The method according to claim 1, wherein in (a),
the genome of the human pluripotent stem cells is modified by fusing a polynucleotide encoding HLA-G1 with an endogenous B2M gene in human pluripotent stem cells, so as to express a B2M-HLA-G1 fusion protein and not express free B2M protein; preferably, an encoding gene of HLA-G1 is introduced into the position before the stop codon of exon 3 of the endogenous B2M gene, or the stop codon in exon 3 of the endogenous B2M gene is replaced by an encoding gene of HLA-G1; more preferably, the modification is performed using gene editing methods; and
an exogenous polynucleotide encoding B2M-HLA-G5 fusion protein is introduced into human pluripotent stem cells, wherein the B2M-HLA-G5 fusion protein comprises B2M and HLA-G5.

3. The method according to claim 1, wherein, a recombinant vector is used to express the B2M-HLA-G1 and B2M-HLA-G5; preferably, the recombinant vector comprises a non-viral vector or a viral vector.

4. The method according to claim 3, wherein the non-viral vector comprises a site-directed knock-in system or a transposon system; or the viral vector comprises a lentiviral vector.

5. The method according to claim 2, wherein the B2M-HLA-G1 fusion protein further comprises a flexible linker peptide between B2M and HLA-G1, preferably the flexible linker peptide has a sequence as shown in SEQ ID NO: 3; and/or the B2M-HLA-G1 fusion protein sequentially comprises B2M and HLA-G1 from the N-terminus to the C-terminus; preferably, the amino acid sequence of the B2M-HLA-G1 fusion protein is as shown in SEQ ID NO: 4; more preferably, the nucleic acid sequence of the B2M-HLA-G1 fusion protein is as shown in SEQ ID NO: 9; and/or
the B2M-HLA-G5 fusion protein further comprises a flexible linker peptide between B2M and HLA-G5, preferably the flexible linker peptide has a sequence as shown in SEQ ID NO: 3; and/or the B2M-HLA-G5 fusion protein sequentially comprises B2M and HLA-G5 from the N-terminus to the C-terminus; preferably, the amino acid sequence of the B2M-HLA-G5 fusion protein is as shown in SEQ ID NO: 8; more preferably, the nucleic acid sequence of the B2M-HLA-G5 fusion protein is as shown in SEQ ID NO: 10.

6. The method according to claim 1, wherein in (b), exon 3 of the CIITA gene in the genome of human pluripotent stem cells is knocked out; preferably, the knockout is performed by gene editing methods; more preferably, a gRNA with the nucleotide sequence shown in SEQ ID NO: 11 is used for gene editing.

7. The method according to any one of the claims 1-6, wherein, it comprises one or more of the following A-D:
A. the HLA-G1 comprises: a peptide with the amino acid sequence as shown in SEQ ID NO: 1; or a derivative peptide having more than 90% sequence identity to SEQ ID NO: 1 and the function of the peptide with the amino acid sequence as shown in SEQ ID NO: 1;
B. the B2M comprises: a peptide with the amino acid sequence as shown in SEQ ID NO: 2; or a derivative peptide having more than 90% sequence identity to SEQ ID NO: 2 and the function of the peptide with the amino acid sequence as shown in SEQ ID NO: 2;
C. the CIITA comprises: a peptide with the amino acid sequence as shown in SEQ ID NO: 5; or a derivative peptide having more than 90% sequence identity to SEQ ID NO: 5 and the function of the peptide with the amino acid sequence as shown in SEQ ID NO: 5;
D. the HLA-G5 comprises: a peptide with the amino acid sequence as shown in SEQ ID NO: 6; or a derivative peptide having more than 90% sequence identity to SEQ ID NO: 6 and the function of the peptide with the amino acid sequence as shown in SEQ ID NO: 6.

8. An immunocompatible human pluripotent stem cell, wherein it (a) does not express free B2M protein but expresses HLA-G1 and secreted HLA-G5; and (b) does not express CIITA protein; preferably, the human pluripotent stem cell comprises an endogenous B2M gene in the genome fusion with a polynucleotide encoding HLA-G1; comprises an exogenous nucleotide encoding a B2M-HLA-G5 fusion protein; and a deleted CIITA gene in the genome.

9. The immunocompatible human pluripotent stem cell according to claim 8, wherein, it is constructed by the method according to any one of the claims 1-5.

10. (canceled)

11. A method for preparing cells suitable for transplantation, comprising: (a) preparing immunocompatible human pluripotent stem cells by the method according to any one of the claims 1-7; (b) further inducing differentiation of the cells in (a) to obtain cells suitable for transplantation.

12. The method according to claim 11, wherein the tissue or organ cells are cardiomyocytes, prepared using CHIR99021 and IWR-1 sequential induction; or the tissue or organ cells are endothelial cells, prepared using CHIR99021, bFGF, VEGF+BMP4 sequential induction.

13. A cardiomyocyte or endothelial cell, derived from immunocompatible human pluripotent stem cells prepared by the method according to any one of the claims 1-7 or immunocompatible human pluripotent stem cells according to claim 8 or 9.

14. (canceled)

15. The method according to claim 10, wherein the cells suitable for transplantation are tissue or organ cells; more preferably, the tissue or organ cells comprise: cardiovascular progenitor cells, cardiomyocytes, endothelial cells, smooth muscle cells, neuronal cells, hematopoietic stem cells, myeloid cells, lymphoid cells, retinal pigment epithelial cells, pancreatic-cells, liver cells, keratinocytes, skeletal muscle cells, adipocytes, osteocytes, chondrocytes, mesenchymal stem cells; more preferably, the myeloid cells comprise granulocytes, monocytes, macrophages, erythrocytes, platelets, and/or the lymphoid cells comprise natural killer cells, T-cells, B-cells, and/or the liver cells comprise hepatocytes, cholangiocytes, liver endothelial cells, hepatic stellate cells, Kupffer cells, mesothelial cells.
